(19) United States
(12) Patent Application Publication
(10) Pub. No.: US 2024/0216959 A1
(43) Pub. Date: Jul. 4, 2024

(54) SYSTEMS, APPARATUSES, AND METHODS UTILIZING A CLEANING FIXTURE

(71) Applicant: Assurant, Inc., New York, NY (US)

(72) Inventor: Brandon Ross, Manchester, TN (US)

(21) Appl. No.: 18/397,349

(22) Filed: Dec. 27, 2023

Related U.S. Application Data
(60) Provisional application No. 63/436,293, filed on Dec. 30, 2022.

(51) Int. Cl. B08B 3/04; A61L 2/18; A61L 101/34; B08B 11/02; H04R 1/10

(52) U.S. Cl. CPC B08B 3/04; A61L 2/18; B08B 11/02; H04R 1/1058; A61L 2101/34; A61L 2202/17

(57) ABSTRACT

Embodiments of the present disclosure provided herein include systems, apparatuses, and methods for utilizing cleaning fixtures. The utilization of cleaning fixtures may be to clean one or more devices and/or device cases. In some embodiments the fixtures may be utilized in a system or apparatus having a plurality of stations, including a pairing station, scanning station, cleaning station, and testing station.

### CROSS-REFERENCE TO RELATED APPLICATIONS

This application claims the benefit of U.S. Provisional Application Ser. No. 63/436,293, filed Dec. 30, 2022, which application is incorporated by reference herein in its entirety.

### TECHNOLOGICAL FIELD

Example embodiments of the present disclosure relate generally to utilizing a cleaning fixture for cleaning devices, including but not limited to cleaning wireless earbuds.

### BACKGROUND

Electronic devices, including wireless electronic devices, have become ubiquitous, and at various times in the lifecycle of a device it may need to be cleaned. Cleaning may extend the lifetime of the device, improve the health of the user, and improve performance of the device. For example, earbuds are common devices used with mobile phone devices to allow a user to enjoy audio conveniently and easily. However, earbuds and other electronic devices may become damaged or dirty with use, including buildup of materials (e.g., earwax, etc.) that may degrade the performance of the earbuds. Many users may trade-in or dispose of the earbuds as they get used or as replacements are purchased. Despite usage or age, the original devices, such as earbuds, may be used again if cleaned and determined to be in working order. Current cleaning processes are rudimentary, including involving individuals using manual, physical tools such as brushes to clean devices. These processes rely on an individual to perform cleaning and also lack a standardized, repeatable, scalable manner of cleaning and testing a device, including but not limited to earbuds, that results in achieving acceptable levels cleanliness, sterilization, and/or performance.

Applicant has identified a number of deficiencies and problems associated with present systems, apparatus, and methods for analyzing networks. Through applied effort, ingenuity, and innovation, many of these identified problems have been solved by developing solutions that are included in embodiments of the present disclosure, many examples of which are described in detail herein.

### BRIEF SUMMARY

Various embodiments described herein relate to systems, apparatuses, and methods for utilizing cleaning fixtures.

In some example embodiments, a fixture configured for cleaning a device is provided. The fixture may comprise at least one fixture body configured to define an inner cleaning cavity bounded by at least one inner cleaning cavity wall. The at least one inner cleaning cavity wall may define a plurality of cleaning cavity ports. The at least one inner cleaning cavity wall may define an output opening. The inner cleaning cavity may be configured to receive a cleaning fluid via the plurality of cleaning cavity ports and permit the cleaning fluid to exit the inner cleaning cavity via the output opening.

In some embodiments, the at least one fixture body may include a first fixture body and a second fixture body configured to engage each other. The at least one inner cleaning cavity wall may include a first inner cleaning cavity wall defined by the first fixture body and a second inner cleaning cavity wall defined by the second fixture body. The inner cleaning cavity may include a first portion and a second portion, the first inner cleaning cavity wall may at least partly bound the first portion of the inner cleaning cavity and the second inner cleaning cavity wall may at least partly bound the second portion of the inner cleaning cavity.

In some embodiments, the plurality of cleaning cavity ports comprises a first plurality of cleaning cavity ports defined by the first inner cleaning cavity wall of the first fixture body, and a second plurality of cleaning cavity ports defined by the second inner cleaning cavity wall of the second body.

In some embodiments, each of the first plurality of cleaning cavity ports is fluidically connected to an exterior of the fixture via at least one first external input port, and each of the second plurality of cleaning cavity ports is fluidically connected to an exterior of the fixture via at least one second external input port.

In some embodiments, the first fixture body comprises a first fluid distribution network configured to fluidically connect each of the first plurality of cleaning cavity ports with the at least one first external input port. The first fluid distribution network may include a first input chamber connected to the at least one first external input port and a plurality of first distribution channels, and the plurality of first distribution channels may be connected to the first plurality of cleaning cavity ports. The second fixture body may include a second fluid distribution network configured to fluidically connect each of the second plurality of cleaning cavity ports with the at least one second external input port. The second fluid distribution network may include a second input chamber connected to the at least one second external input port and a plurality of second distribution channels, and the plurality of second distribution channels may be connected to the second plurality of cleaning cavity ports.

In some embodiments, the output opening of the inner cleaning cavity is defined between the first fixture body and the second figure body in an instance in which the first fixture body and the second fixture body are engaged with each other.

In some embodiments, the output opening and each of the plurality of cleaning cavity ports are configured to be smaller than the device to prevent the device from leaving the inner cleaning cavity in the instance in which the first fixture body and the second fixture body are engaged with each other.

In some embodiments, the output opening is defined circumferentially around the inner cleaning cavity.

In some embodiments, the fixture further comprises an output port configured to fluidically couple the output opening to an exterior of the fixture.

In some embodiments, the first inner cleaning cavity wall defines a first shape that is configured to match a first half of the device with a clearance between the device and the first inner cleaning cavity wall. The second inner cleaning cavity wall may define a second shape that is configured to match a second half of the device with a clearance between the device and the second inner cleaning cavity wall.

In some embodiments, the first fixture body further comprises one or more first guides and the second fixture body comprises one or more second guides, and the first guides comprise complementary shapes to the associated second guides to laterally align the first fixture body with the second fixture body.

In some embodiments, the first fixture body defines a first external wall, the second fixture body defines a second external wall, and the first fixture body and second fixture body are configured to be mated to form an airtight seal between the first external wall and second external wall.

In some embodiments, the at least one fixture body is further configured to define an outer cavity separate from the inner cleaning cavity and fluidically coupled to the inner cleaning cavity via the output opening.

In some embodiments, the outer cavity is fluidically connected to an exterior of the fixture via an output port.

In some embodiments, the outer cavity is configured to surround the inner cleaning cavity along at least one plane.

In some embodiments, the at least one fixture body comprises a first fixture body and a second fixture body configured to engage each other at the at least one plane, and the output opening is defined between the first fixture body and the second fixture body.

In some embodiments, a method for cleaning a device using a fixture is provided. The fixture may include at least one fixture body configured to define an inner cleaning cavity bounded by at least one inner cleaning cavity wall. The at least one inner cleaning cavity wall may define a plurality of cleaning cavity ports. The at least one inner cleaning cavity wall may define an output opening. The inner cleaning cavity may be configured to receive a cleaning fluid via the plurality of cleaning cavity ports and permit the cleaning fluid to exit the inner cleaning cavity via the output opening. The method may include placing the device into the inner cleaning cavity of the fixture, and cleaning the device by: introducing the cleaning fluid into the inner cleaning cavity via each of the plurality of cleaning cavity ports; and allowing the cleaning fluid to exit the inner cleaning cavity via the output opening.

In some embodiments, the method further comprises introducing air into the fixture via each of the plurality of cleaning cavity ports, and allowing the air to leave the inner cleaning cavity via the output opening.

In some embodiments, allowing the air to leave the inner cleaning cavity via the output opening comprises extracting, via a vacuum generated by a pump, air from the inner cleaning cavity.

In some embodiments, the cleaning fluid includes ethyl alcohol.

In some embodiments, introducing the cleaning fluid is for a first period of time, and allowing the cleaning fluid to exit the inner cleaning cavity occurs after an end of the first period of time.

In some embodiments, the at least one fixture body includes a first fixture body and a second fixture body.

In some embodiments, the first fixture body comprises one or more guides, and the second fixture body comprises one or more guide cavities, wherein each of the one or more guide cavities is configured to mate to one of the guides of the first fixture body.

In some embodiments, the method further comprises mating, prior to the cleaning, the first fixture body to the second fixture body utilizing the one or more guides and one or more guide cavities.

In some embodiments, the at least one inner cleaning cavity wall includes a first inner cleaning cavity wall and a second inner cleaning cavity wall, the plurality of cleaning cavity ports includes a first plurality of cleaning cavity ports defined by the first inner cleaning cavity wall and a second plurality of cleaning cavity ports defined by the second inner cleaning cavity wall, the first fixture body comprises the first inner cleaning cavity wall and at least one external input port of the first fixture body fluidically connected to the first plurality of cleaning cavity ports, and the second fixture body comprises the second inner cleaning cavity wall and at least one external input port of the second fixture body fluidically connected to the second plurality of cleaning cavity ports.

In some embodiments, introducing the cleaning fluid into the inner cleaning cavity via each of the plurality of cleaning cavity ports comprises introducing cleaning fluid via the at least one external input port of the first fixture body and via the at least one external input port of the second fixture body.

In some embodiments, a system for cleaning devices is provided. The system may include a fixture comprising, at least one fixture body configured to define an inner cleaning cavity bounded by at least one inner cleaning cavity wall, at least one external input port. The at least one inner cleaning cavity wall may define a plurality of cleaning cavity ports. The at least one inner cleaning cavity wall may define an output opening. The inner cleaning cavity may be configured to receive a cleaning fluid via the plurality of cleaning cavity ports and permit the cleaning fluid to exit the inner cleaning cavity via the output opening. The system further may include a fluid supply connected to the at least one external input port and an air supply connected to the at least one external input port.

In some embodiments, the system further comprises a cleaning station configured to connect the air supply and the fluid supply to the fixture.

In some embodiments, the fixture comprises at least one fixture body including a first fixture body and a second fixture body. The first fixture body and the second fixture body may be configured to engage each other. The cleaning station is further configured to engage the first fixture body with the second fixture body, and connect the air supply and the fluid supply with the fixture only after engagement of the first fixture body with the second fixture body.

In some embodiments, the system further comprises a pairing station configured to pair to at least one device.

In some embodiments, the pairing station comprises at least one actuator to actuate one or more buttons associated with a device to pair the device to the system.

In some embodiments, the system further comprises a scanning station configured to scan a device identifier of a device.

In some embodiments, the system further comprises at least one robotic arm configured to position a device identifier in view of an optical reader.

In some embodiments, the system further comprises a testing station configured to test a device.

In some embodiments, the device comprises an audio device. The testing station may include one or more microphones. The testing station may be configured to: generate at least one audio signal to be emitted by the audio device; transmit the at least one audio signal to the audio device; generate, by the one or more microphones and in response to the at least one audio signal being emitted by the audio device, at least one electrical signal associated with the audio device; and generate a grading of the audio device based on the at least one electrical signal.

In some embodiments, the system further comprises one or more rotary index tables configured to rotate a device to one or more stations.

In some embodiments, the one or more stations include one or more of a pairing station, a scanning station, and a testing station.

In some embodiments, a method for cleaning and testing a device is provided, and the method comprises cleaning the device using a fixture. The fixture may include at least one fixture body configured to define an inner cleaning cavity bounded by at least one inner cleaning cavity wall. The at least one inner cleaning cavity wall may define a plurality of cleaning cavity ports. The at least one inner cleaning cavity wall may define an output opening. The inner cleaning cavity may be configured to receive a cleaning fluid via the plurality of cleaning cavity ports and permit the cleaning fluid to exit the inner cleaning cavity via the output opening. The method further may include cleaning the device using the fixture comprises: placing the device into the inner cleaning cavity of the fixture; introducing the cleaning fluid into the inner cleaning cavity via each of the plurality of cleaning cavity ports; and allowing the cleaning fluid to exit the inner cleaning cavity via the output opening. The method further may include testing the device, after cleaning the device and via at least one testing station, to generate a grading of the performance of the device.

In some embodiments, allowing the cleaning fluid to exit the inner cleaning cavity via the output opening comprises introducing air from an air supply into the inner cleaning cavity via each of the plurality of cleaning cavity ports.

In some embodiments, the at least one fixture body includes a first fixture body and second fixture body, wherein the first fixture body comprises one or more guides, wherein the second fixture body comprises one or more guide cavities, wherein each of the one or more guide cavities is configured to mate to one of the guides of the first fixture body. The method may further include mating, after the placing the device into the inner cleaning cavity of the fixture and before connecting a fluid supply to the fixture, the first fixture body to the second fixture body utilizing the one or more guides and one or more guide cavities.

In some embodiments, the device is an audio device. The testing station may include one or more microphones. Testing the device further may include: generating at least one audio signal to be emitted by the audio device; transmitting the at least one audio signal to the audio device; generating, by the one or more microphones and in response to the at least one audio signal being emitted by the audio device, at least one electrical signal associated with the audio device; and generating the grading of the audio device based on the at least one electrical signal.

In some embodiments, the method further comprises pairing, before cleaning the device, the device with a pairing station.

In some embodiments, the method further comprises scanning a device identifier of the device with a scanning station.

In some embodiments, scanning a device identifier comprises: positioning the device identifier of the device in a view of an optical reader with at least one robotic arm; and scanning, with the optical reader, the device identifier.

In some embodiments, the method further comprises generating an acceptance indication based on the grading, and sorting the device based on the acceptance indication.

In some embodiments, the sorting the device utilizes at least one robotic arm to sort the device to one of a plurality of locations.

The above summary is provided merely for purposes of summarizing some example embodiments to provide a basic understanding of some aspects of the disclosure. Accordingly, it will be appreciated that the above-described embodiments are merely examples and should not be construed to narrow the scope or spirit of the disclosure in any way. It will also be appreciated that the scope of the disclosure encompasses many potential embodiments in addition to those here summarized, some of which will be further described below.

### BRIEF SUMMARY OF THE DRAWINGS

Having thus described certain example embodiments of the present disclosure in general terms, reference will now be made to the accompanying drawings, which are not necessarily drawn to scale, and wherein:

FIG. 1A illustrates an example cleaning and testing system utilizing a fixture in accordance with some embodiments of the present disclosure;

FIG. 1 B illustrates an embodiment of an example cleaning and testing system utilizing a fixture in accordance with the present disclosure;

FIG. 2 illustrates an example of a charging cradle in accordance with some embodiments of the present disclosure;

FIG. 3 illustrates an example of loading tray in accordance with some embodiments of the present disclosure;

FIG. 4 illustrates an example block diagram of a cleaning station in accordance with some embodiments of the present disclosure;

FIG. 5A illustrates an example first fixture body from a first perspective in accordance with some embodiments of the present disclosure;

FIG. 5B illustrates an example first fixture body from a second perspective in accordance with some embodiments of the present disclosure;

FIG. 6A illustrates an example second fixture body from a first perspective in accordance with some embodiments of the present disclosure;

FIG. 6B illustrates an example second fixture body from a second perspective in accordance with some embodiments of the present disclosure;

FIG. 7 illustrates an example of a fixture in accordance with some embodiments of the present disclosure;

FIG. 8 illustrates an example fluid distribution network of a fixture in accordance with some embodiments of the present disclosure;

FIG. 9A illustrates an example of a loading tray and a loading tray lid in accordance with some embodiments of the present disclosure;

FIG. 9B illustrates an example of a loading tray lid in accordance with some embodiments of the present disclosure;

FIG. 9C illustrates an example of a loading tray in accordance with some embodiments of the present disclosure;

FIG. 9D illustrates a first cross-section of an example of a fixture in accordance with some embodiments of the present disclosure;

FIG. 9E illustrates a second cross-section an example of a fixture in accordance with some embodiments of the present disclosure;

FIG. 9F illustrates a first perspective of an example of a cleaning station in accordance with some embodiments of the present disclosure;

FIG. 9G illustrates a first perspective of an example of a cleaning station in accordance with some embodiments of the present disclosure;

FIG. 9H illustrates a third perspective of a cross section of an example of a loading tray and loading tray lid in a cleaning station in accordance with some embodiments of the present disclosure;

FIGS. 9I and 9J illustrate an example of a testing station in accordance with some embodiments of the present disclosure;

FIG. 10 illustrates an example process for cleaning and testing a device in accordance with some embodiments of the present disclosure;

FIG. 11 illustrates an example pairing process in accordance with some embodiments of the present disclosure;

FIG. 12 illustrates an example scanning and process in accordance with some embodiments of the present disclosure;

FIG. 13 illustrates an example cleaning process in accordance with some embodiments of the present disclosure;

FIG. 14 illustrates an example testing process in accordance with some embodiments of the present disclosure;

FIG. 15 illustrates an example unloading process in accordance with some embodiments of the present disclosure; and

FIG. 16 illustrates an example computing device in accordance with some embodiments of the present disclosure.

### DETAILED DESCRIPTION

Some embodiments of the present disclosure will now be described more fully herein with reference to the accompanying drawings, in which some, but not all, embodiments of the disclosure are shown. Indeed, various embodiments of the disclosure may be embodied in many different forms and should not be construed as limited to the embodiments set forth herein; rather, these embodiments are provided so that this disclosure will satisfy applicable legal requirements. Like reference numerals refer to like elements throughout.

As used herein, the phrases “in one embodiment,” “according to one embodiment,” “in some embodiments,” and the like generally refer to the fact that the particular feature, structure, or characteristic following the phrase may be included in at least one embodiment of the present disclosure. Thus, the particular feature, structure, or characteristic may be included in more than one embodiment of the present disclosure such that these phrases do not necessarily refer to the same embodiment or preclude different features, structures, or characteristics from being included in the same and/or different embodiments.

As used herein, the word “example” or “exemplary” is used herein to mean “serving as an example, instance, or illustration.” Any implementation described herein as “example” or “exemplary” is not necessarily to be construed as preferred or advantageous over other implementations. As used herein, the term “or” is used in both the alternative and conjunctive sense, unless otherwise indicated.

The figures are not drawn to scale and are provided merely to illustrate some example embodiments of the inventions described herein. The figures do not limit the scope of the present disclosure or the appended claims. Several aspects of the example embodiments are described below with reference to example applications for illustration. It should be understood that numerous specific details, relationships, and methods are set forth to provide a full understanding of the example embodiments. One having ordinary skill in the relevant art, however, will readily recognize that the example embodiments can be practiced without one or more of the specific details or with other methods. In other instances, well-known structures and/or operations are not shown in detail to avoid obscuring the example embodiments.

### Overview

Various embodiments of the present inventions disclosed herein are directed to improved systems, apparatuses, and methods utilizing a cleaning fixture. A cleaning fixture may be referred to herein as a fixture.

Various embodiments include a fixture for cleaning devices, including but not limited to electronic devices such as wireless devices (e.g., wireless earbuds, mobile consumer electronic devices, watches, smart phones, mobile computers, tablet computers, wearables, hearables, handheld electronic devices, and the like). Electronic devices, including wireless devices, have become commonplace but often a user discards or trades in a wireless device before the wireless device has reached the end of its useable life. Indeed, the pace of introduction of new technology progresses quickly, which has created large amounts of devices that are still useful but may have been discarded. Before such devices may be passed from one user to another, they may need to be cleaned and tested to ensure they are in good working order and/or to address any resolvable issues with the device (e.g., removing dirt and debris, repairing malfunctioning parts, or the like). This need has been made further apparent by health pandemics, which has heightened concern about receiving devices used by others. The present disclosure includes multiple improvements described herein, including improved cleaning of devices to achieve acceptable levels of cleanliness, sterilization, and/or performance.

In various embodiments, the system, apparatuses, and method disclosed herein may utilize one or more cleaning fixtures, also referred to herein as fixtures. The fixtures may be configured to clean a device, such as having one or more cavities configured for a device as well as one or more ports for introducing cleaning fluid(s) and air into contact with the device. For example, various embodiments herein may be disclosed to clean and test wireless earbuds (e.g., AirPods®, Galaxy Buds®, or the like). The cleaning may include testing the cleaned device to confirm that the device performs at an acceptable level for further use. The testing may be used to generate a grading of the device's performance. The grading may include a quality grade and/or one or more performance levels, which may be based on one or more thresholds and one or more tests. In various embodiments, the cleaning may sanitize the device. For example, the cleaning and/or sanitization in accordance with various embodiments described herein may remove 99.9% or more of the coronavirus disease 2019 (a.k.a., COVID-19). In some embodiments, a system may be used to clean two or more devices simultaneously or sequentially to repeatably and dependably clean and test devices with little to no human interaction. As used herein, the term “device” may be inclusive of any number of devices without limitation unless specified otherwise. For example, a pair of earbuds may be considered a single device or multiple devices depending on the context, and a chargeable case associated with the earbuds may be considered part of the same device or a third device again depending on the context.

A cleaned device that may need to be determined to be at an acceptable performance level may be transmitted to a third party (e.g., repurposed or resold). Alternatively, a device determined not to perform at an acceptable level or a received a certain grade may be discarded. In various embodiments, a discarded device may be recycled.

As a non-limiting illustrative example, an initial user may trade in a pair of wireless earbuds, such as at a retailer (e.g., Best Buy®, Target®, or the like). While the earbuds may be traded in due to the initial user no longer intending to use them, the earbuds may still be useful to another user. Instead of discarding the earbuds as waste, the earbuds might be refurbished for use by another user. But before this other user may receive the earbuds, cleaning and confirming the earbuds perform at an acceptable level may be required. After cleaning and testing the earbuds might be resold based on one or more acceptable performance levels, such as new or used. To clean the earbuds, a fixture as disclosed herein may be used to assist with the cleaning in a standardized manner that achieves a desired level of cleaning on a consistent basis. In other embodiments, damaged and/or defective devices may be transmitted for cleaning and testing, and/or a user may use or transmit a device to a facility for cleaning and testing in the normal course of upkeep of the device (e.g., to be returned to the user for further use).

It should be readily appreciated that the embodiments of the systems, apparatus, and methods described herein may be configured in various additional and alternative manners in addition to those described herein.

### Definitions

As used herein, the terms “data,” “content,” “information,” and similar terms may be used interchangeably to refer to data capable of being transmitted, received, displayed, and/or stored in accordance with embodiments of the present disclosure. Thus, use of any such terms should not be taken to limit the spirit and scope of embodiments of the present disclosure. Further, where a system or user device is described herein to receive data from another system or user device, it will be appreciated that the data may be received directly from another user device or may be received indirectly via one or more intermediary user devices, such as, for example, one or more systems, servers, relays, routers, network access points, base stations, hosts, and/or the like, sometimes referred to herein as a “network.” Similarly, where a system or a user device is described herein to send data to another user device or system, it will be appreciated that the data may be sent directly or may be sent indirectly via one or more intermediary systems or user devices, such as, for example, one or more systems, servers, relays, routers, network access points, base stations, hosts, and/or the like.

As used herein, the term “circuitry” refers to (a) hardware-only circuit implementations (e.g., implementations in analog circuitry and/or digital circuitry); (b) combinations of circuits and computer program product(s) comprising software and/or firmware instructions stored on one or more computer readable memories that work together to cause an apparatus to perform one or more functions described herein; and (c) circuits, such as, for example, a microprocessor(s) or a portion of a microprocessor(s), that require software or firmware for operation even if the software or firmware is not physically present. This definition of “circuitry” applies to all uses of this term herein, including in any claims. As a further example, as used herein, the term “circuitry” also includes an implementation comprising one or more processors and/or portion(s) thereof and accompanying software and/or firmware. As another example, the term “circuitry” as used herein also includes, for example, an integrated circuit or applications processor integrated circuit for a mobile phone or a similar integrated circuit in a server, a cellular network device, system, other network device, and/or other computing device.

As used herein, a “computer-readable storage medium,” which refers to a physical storage medium (e.g., volatile or non-volatile memory device), may be differentiated from a “computer-readable transmission medium,” which refers to an electromagnetic signal. As used herein, the term “data object” refers to electronically managed data capable of being collectively transmitted, received, and/or stored. A data object may be defined by one or more associated data structures, which may include at least a data structure identifier that uniquely identifies a data structure represented by the data object and/or the data object itself. For example, a data object may refer to a collection of data and instructions that represent data of station and/or data of a plurality of station. For example, a data object may comprise one or more data structures defined by data properties.

### Example System and Apparatuses

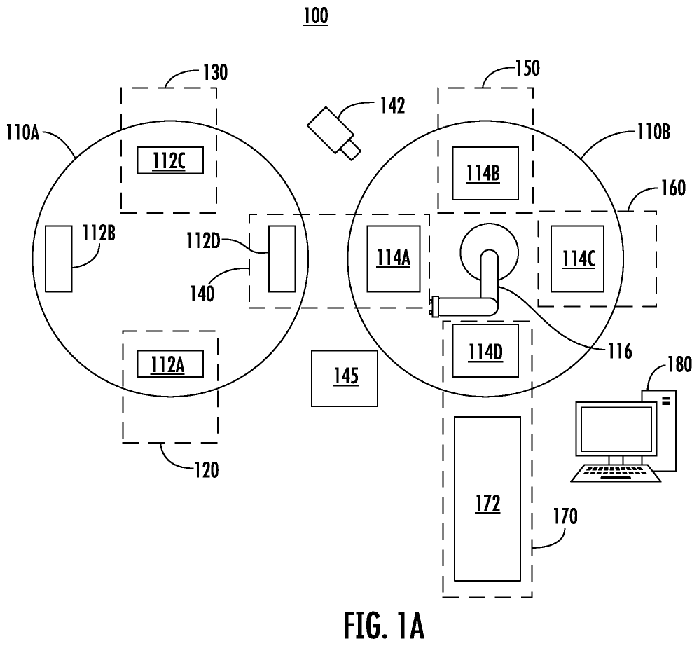

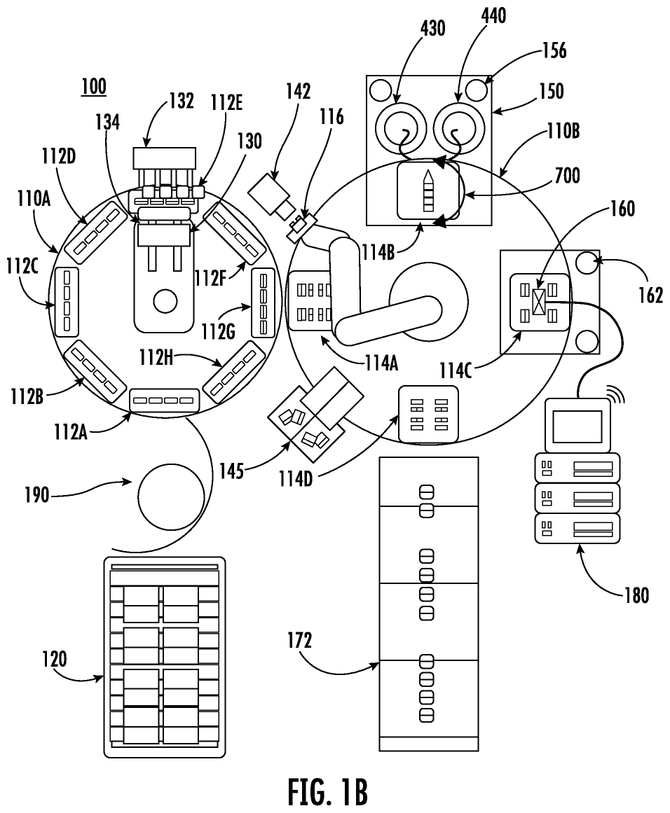

FIG. 1A illustrates an example cleaning and testing system 100 that may utilize a fixture in accordance with some embodiments of the present disclosure. The cleaning and testing system 100 may include one or more stations, components, fixtures, or the like described herein and may be configured in many different ways. While the various embodiments described herein may include one or more examples of cleaning and testing systems, including how the cleaning and testing system 100 may be embodied, it is readily appreciated that the various stations, components, and/or fixture are also usable independently and in any possible combinations or sub-combinations.

The cleaning and testing system 100 may include, among other things, one or more of a loading station 120, a pairing station 130, a scanning station 140, a reject station 145, a cleaning station, 150, a testing station 160, and/or an offloading station 170. The cleaning and testing system 100 may include a computing device 180 that may allow a user to interface with the cleaning and testing system 100. In various embodiments, each station may include its own computing device 180 that may control or operate the station, and the station computing devices 180 may be in communication with a system computing device 180. In some embodiments, the computing device(s) may control the functionality of the various system components described herein. A cleaning and testing system 100 may include one or more index tables, such as index table 110A and index table 110B, which may rotate or move a device to be cleaned and tested through the stations of a cleaning and testing system 100. The cleaning and testing system 100 may also utilize one or more charging cradles 112 and/or one or more loading trays 114.

An index table 110A, 110B may be a rotary index table that may rotate a device between some or all of the stations of a cleaning and testing system 100. Alternatively, an index table 110 may move one or more devices between multiple indexed positions associated with one or more of the stations. While FIG. 1A illustrates two index tables, a single index table 110 may be used with each station having a respective location on this index table. Alternatively, more than two index tables may be used, such as when a cleaning and testing system 100 may have multiple of the same station being operated in parallel. For example, if one station takes longer to operate than other stations then there may be multiple versions of certain stations. In some embodiments, any other type of device holding platform(s) may be used, such as conveyors, fixed tables, racks, or the like.

In various embodiments, an index table 110A, 110B may include or one more charging cradles 112. For example, there may be four charging cradles, such as a first charging cradle 112A, second charging cradle 112B, third charging cradle 112C, and a fourth charging cradle 112D. Various embodiments may have more or less charging cradles 112 that may be moved between stations. In some embodiments, the number of charging cradles may be equal to the number of indexed positions of a corresponding index table for embodiments utilizing a rotary index table.

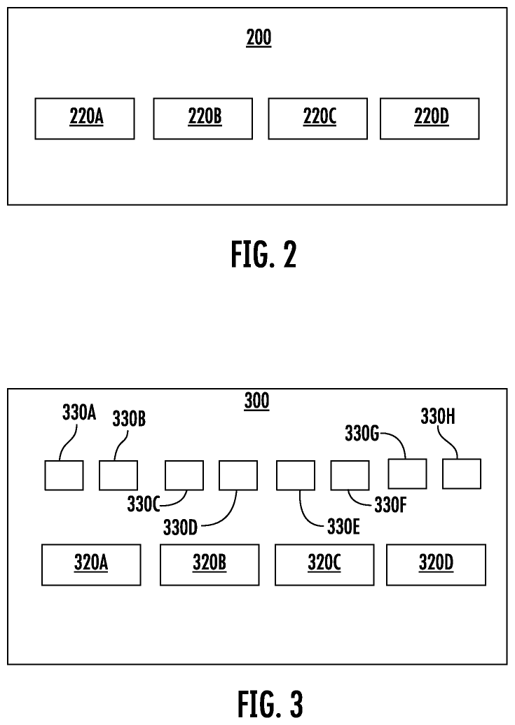

Many devices (e.g., earbuds) may need to be charged before they can be operated, including operated for testing (e.g., devices may be sent in on an empty battery). A charging cradle 112 may be used to charge one or more devices. A charging cradle 112 may charge a device based on the device's charging method, such as a wired or wireless connection. The charging cradle 112 may be wired to an index table 110 or may be wireless connected to an index table 110 to wirelessly power the charging cradle 112. Charging cradles 112 are described further herein, including in relation to FIG. 2. In some embodiments, the charging cradles may be supplied power to charge the device(s) during at least a portion of the cleaning and testing process.

In the embodiment of FIG. 1A, a charging cradle 112 may be indexed between four or more positions. In this embodiments, the index table 110A includes four charging cradles 112A, 112B, 112C, and 112D, each illustrated at one of four different positions. The index table 110A illustrated rotates, for example, a charging cradle 112A through each of the positions illustrated from charging cradles 112B, 112C, and 112D before returning to the positions illustrated for charging cradle 112A. The timing of moving a charging cradle 112 through each of the positions, including to each of the stations, may be controlled for one or more time periods. For example, the movement of the charging cradle 112 may include a first time period to allow for a minimum charge to be put on a device before the device is moved to a first station, such as a pairing station 120. The cleaning and testing system 100 may, on determining or receiving an identification of the type of device, adjust the time periods, including to allow for a minimum charge to be put onto a device. The time period may, for example, be based on an average time to charge a device.

A loading station 120 may facilitate the loading of one or more devices into the cleaning and testing system 120, such as into a charging cradle 112A or another holding device. The loading station 120 may be used by an operator (e.g., a human operator or robotic device), who may receive one or more of the devices and individually load devices into the cleaning and testing system 120. The loading station 120 may include one or more robots that may assist with or perform the loading of one or more devices into the cleaning and testing station 100, such as with one or more robotic arms 116. In various embodiments, one or more robotic arms 116 may be used to place one or more devices into a charging cradle 112 or otherwise onto one or more portions of the cleaning and testing system 100. The loading station 120 may receive packages containing the inbound device(s), such as via a conveyor, and may facilitate the operator removing each device and placing the devices individually in one or more corresponding locations in the charging cradle. For example, earbuds may be left inside a charging case and the entire charging case, with earbuds inside, may be placed in a receiving location on the charging cradle. In the depicted embodiment, the index table 110A may then rotate clockwise towards a pairing station 130. In some embodiments, additional delay may be introduced to allow greater charge time by rotationally offsetting the pairing station 130 from the loading station 120 and/or by adding additional charging cradles to allow more charge time between loading and pairing (e.g., eight cradles instead of four). Similarly, the number of devices held by each cradle may additionally or alternatively be selected (e.g., four devices per cradle).

A pairing station 130 may provide for pairing a device with the cleaning and testing system 100. Various embodiments may include one or more robots or actuators to operate one or more devices to pair the devices with the cleaning and testing system 100. The cleaning and testing station 100 may generate a pairing determination, which may indicate if pairing was successful or not. If a pairing determination indicates that pairing was not successful then the device(s) and/or device case may be discarded by moving it to rejection area, such as a rejection station 145, which may be done with one or more robotic arms 116 and/or conveyor(s).

For example, when the devices may be earbuds in an earbud case, the pairing station 130 may include one or more actuators to perform a sequence of actions to pair the earbuds. For example, FIG. 1B illustrates a case opening actuator 134 configured to open the lid of a flip-top earbud case and a pairing actuator 132 comprising a plurality of individual actuators configured to press a “pairing” button on the device cases. The pairing station may open the earbud case and actuate one or more buttons on the earbud case to initiate pairing, which may include making the earbuds discoverable to the cleaning and testing system 100, including but not limited to a computing device 180 of the cleaning and testing system 100. The computing device 180 may then have a wireless receiver (e.g., a Bluetooth® receiver) configured to pair with the device(s) during the cleaning and testing.

In various embodiments, the cleaning and testing station 100 may track a device through the cleaning and testing system 100 based on the pairing of the device with the cleaning and testing station 100. During pairing the location of a device is determined for tracking, such as by tracking which actuators are used to actuate one or more buttons associated with a device and mapping the cradle, slot, or other physical location towards which the actuator was moved with the device to which the computing device 180 has just paired. Additionally or alternatively, one or more computer vision systems may be used with the cleaning and testing system 100 to track, identify, test, analyze, and/or otherwise facilitate portions of the operations described herein. As the cleaning and testing system 100 may move the device through one or more stations, the identity and location of the device are tracked, such as by knowing which station a device may be at, which charging cradle 112 a device is in, which loading tray 114 a device is in, or the like as the cleaning and testing station 100 may track the locations of each of these. Such tracking may be performed by the computing device 180. In various embodiments, the pairing station 130 may include one or more robotic arms (e.g., 116), which may comprise one or more of the actuators described.

In various embodiments, during pairing a pairing identifier may be communicated between a device and the cleaning and testing station 100, and the pairing identifier may be used to track a device and/or data associated with the device in the cleaning and testing station 100. For example, a Bluetooth® pairing may include a device sharing a pairing identifier of a MAC address with the cleaning and testing station 100. During subsequent operations, such as those associated with the testing station 160, test results data may be associated with the pairing identifier in addition to other data described herein, such as a identifier associated with the optical reader 142.

A scanning station 140 may provide for scanning of a device identifier associated with a device and/or physical inspection of the device. A device identifier may be an indicia associated with the device, which may be located on a device, a device case associated with the device, or the like. In various embodiments, a device identifier may be a serial number, a bar code, a QR code, or an indicia that may be read that identifies the device. The scanning station may read text from the surface of the device and/or one or more computer-readable indicia from the surface of the device. In some embodiments, the scanning station, via communication with the computing device 180, may confirm electronic data transmitted between the device and the computing device (e.g., an electronic identifier transmitted during or following pairing). A device identifier may be unique to the device. In various embodiments, the unique identifier may be used to identify if an associated device is authentic or if the associated device is not authentic. Such an identification may be determined based on a comparison of a device identifier with one or more device identifiers in an authentication data object that may include a listing of, for example, serial number or the like. An authentication data object may be stored in a memory of a computing device 180.

A scanning station may use an optical reader 142 to read or scan the device identifier. In various embodiments, the optical reader 142 may be a camera. The optical reader 142 may be connected or coupled to a computing device 180. The optical reader 142 may capture an image of a device identifier, and the optical reader 142 may optically recognize or decode a device identifier in the captured image to generate a device identifier data object. In various embodiments, optical recognition may include optical character recognition to convert one or more portions of an image into characters that may be used to authenticate a device. The optical reader 142 may provide the device identifier data object to the computing device 180. Alternatively, or additionally, the image captured by the optical reader 142 may be transmitted to the computing device 180 for decoding or optically recognizing the device identifier.

A scanning station 140 may be associated with a robotic arm 116. The robotic arm 116 may manipulate one or more device(s), including device cases, to position the device or device case in view of the optical reader 142, including to place the device identifier in view of the optical reader 142. In some embodiments, a device (e.g., one or a pair of earbuds) and a device case may be held by the robotic arm 116 and oriented towards the camera for imaging and processing. In some embodiments, the earbuds and case may be held sequentially by the robotic arm. In some embodiments, each earbud may be held alone. In some embodiments, the optical reader 142 may capture an image of the identifying information on the device, such as a serial and/or model number. Through the tracking of a device from the pairing station 130, the scanning station 140 may generate additional data or information, such as images and/or data objects, for tracking the device. For example, in various embodiments one or more device identifiers may be scanned by the scanning station 140 and put in a data object associated with the device that has been generated by the cleaning and testing system 100. This data object may also be used to store testing results.

In various embodiments, the robotic arm 116 may rotate or position the device and/or device case in view of the optical reader 142 for the optical reader 142 to capture one or more images of the device and/or case associated with the device. Such captured images may be used by the cleaning and testing station 100 to determine if there is physical damage to the device and/or case associated with the device. This may be determined by the scanning station 140 analyzing one or more images of the device and/or device case with one or more images of a new device and/or device case to identify differences and/or damage.

Captured images from the optical reader 142 may also be used to grade a device(s) and/or a case associated with a device. In some embodiments, the grades may be established based on a number of criteria and/or a number of output values. In some embodiments, the grades may be categories (e.g., levels) associated with the device(s) or case and/or threshold tests associated with the device(s) or case. In various embodiments, a first grade may be or may be indicative of authentic or not authentic, in which the optical reader 142 and corresponding processing performed by the computing system 180 determine if the imaged device matches an expected device or type/category of expected devices. The comparison may be performed, for example, via a trained machine learning mode. In various embodiments, at least a portion of the grading process may comprise determining a condition grade, which may be on a scale of how damaged or worn a device or case associated with a device may be, such as like new, worn, very worn, damaged, or the like. Based on one or more grading, a cleaning and testing system 100 may generate a determination associated with the device, such as accepting or rejecting a device. In an instance in which a rejection is determined, the cleaning and testing system 100, such as with a robotic arm 116, may place a device and/or a device case into a reject area, such as reject station 145.

In various embodiments, including the illustrated embodiment of FIG. 1A, the scanning station 140 may include a transfer station, which may be a separate station in other embodiments. The scanning station 140 including the transfer station may transfer a device(s) and/or a device case to be transferred from a first index table 110A to a second index table 110B, including from a charging tray 112D to a loading tray 114A. In embodiments using a single table or other platform, the robotic arm or another mechanism may transfer the device(s) and/or a device case between stations on the single platform. The transfer may be performed with a robotic arm 116, which may grasp, lift, transfer, and/or place a device and/or a device case. The loading tray 114 is described further herein, such as in association with FIG. 3. In various embodiments, a cleaning and testing station 100 may use only one loading tray 114. Alternatively, one or more loading trays 114 may be omitted, such as when one or more conveyors may be used.

In various embodiments where a device may be in a device case, a device may be transferred from its device case by a combination of operations at pairing station 130 and scanning station 140. For example, at pairing station 130 one or more actuators may open the device case, which may allow for the device to be removed. At scanning station 140, a robotic arm 116 may transfer the device from the device case to a loading tray 114. In various embodiments, the robotic arm 116 may transfer the device to a fixture located on or in a loading tray 114.

Various embodiments may not include a specific portion of a cleaning and testing station 100 designated as a scanning station 140 (e.g., a designated stop of the first index table 110A), but there may be a portion of a cleaning and testing system 100 associated with the functions and/or operations described for a scanning station 140. For example, the first index table 110A may continuously rotate and a robotic arm 116 may perform the functions and/or operations described herein while the first index table 110A rotates. Alternatively, a robotic arms 116 may perform one or more functions and/or operations described herein with a scanning station 140 while transferring a device from a pairing station 130 to a cleaning station 150. It is readily appreciated that such a portion of the cleaning and testing system 100 where a robotic arm 116 performs the operations and/or functionality described herein associated with a scanning station 140 may be considered a scanning station 140.

In various embodiments, an index table 110 or the like may move a device and/or device case from one station to another. In various embodiments, the device and/or the device case may be loaded onto a tray 114 for movement. In some embodiments, the trays 114 may include charging capabilities. In some embodiments, only the cradles 112 may include charging capabilities. In some embodiments, the device and/or device case may not be loaded onto a tray. An index table 110 may move the device and/or device case to one or more positions, such as a station, which is a set distance or rotation from each position, In embodiments including a rotary index table 110A, the rotary index table may rotate 360 degrees, and the rotation may be in a plurality of increments or may be continuous. The speed and distance of each rotational movement may be controlled by the cleaning and testing system 100, such as by a computing device 180 controlling one or more motors and/or actuators. In various embodiments, two index tables may be used, such as a first rotary index table 110A and a second rotatory index table 110B. Each index table 110 may be associated with one or more stations described herein. In various embodiments, one or more conveyors may be used in combination and/or instead of an index table 110. Similarly, the one or more conveyors may be controlled to move a device and/or device case from any first positions to any second position, such as from a first station to a second stations. Various embodiments may also include a robotic arm 116 which may be used in conjunction with an index table(s) and conveyor(s) to position a device and/or a device case.

Cleaning station 150 may include one or more cleaning apparatus, component, or the like, including utilizing one or more fixtures. The cleaning station 150 and/or fixtures may be used to clean a device and/or a device case. Example embodiments of the cleaning station 150, fixtures, and methods and apparatuses associated therewith are described further herein. With reference to FIG. 1B, the cleaning station 150 is depicted having a cover actuated by one or more lifters 156 to enclose a fixture 700 for cleaning and manipulation. The fixture may be fluidically connected to one or more supplies 430, 440 (e.g., reservoirs or containers) for delivery of fluid and/or gas to the fixture.

Testing station 160 may include one or more test apparatus, sensors, monitors, and/or test equipment (e.g., microphones, speakers, oscilloscopes), and the like, which may be collectively used to perform one or more tests of a device and/or device case. For example, a Python test script may be run to conduct one or more tests of the device and/or device case.

For example, in various embodiments with a device of earbuds, a test station 160 may include, among other things, one or more microphones as well as a computing device, which may be the same computing device for the entire system or one or more dedicated testing computers. The computing device(s), having been paired to the earbuds, may control the earbuds to play one or more sounds. For example, the test station 160 may transmit to the earbuds one or more sounds at different frequencies to test the output of the earbuds (e.g., via a speaker located adjacent the tray 114C). In the embodiment depicted in FIG. 1B, the testing station 160 includes an enclosure controlled by one or more lifters 162 to surround the device(s) and/or case(s) for testing (e.g., to acoustically and/or optically isolate the device(s) and/or case(s) from an external environment. The test station 160 may use the one or more microphones, which may be placed in one or more specific locations in relation to the device being tested. In some embodiments, the microphones and the device(s) and/or device case(s) may be placed in an enclosure to isolate the microphones and respective device(s) and/or device case(s) from the external environment during testing. The one or more microphones may be referred to as a microphone array in some embodiments. On an emitted a sound, the one or more microphones may receive the sound(s) and convert them to one or more electrical signals indicative of the sound(s). The test station 160 may measure the one or more electrical signals indicative of the sound(s) and may compare them against a predetermined threshold and/or a predetermined range to grade the earbuds. The grading may, for example, include a frequency as well as a volume level. The sound of a frequency may be a frequency sweep, which may be kept at a single volume level or may have the volume varied. The test station 160 may iterate one or more tests before assigning a grade, which may be set, for example, according to one or more thresholds associated with the test results. If the earbuds receive a passing grade, the passing grade may be transmitted to the computing entity 180, such as in or with one or more data objects, to update records or data objects stored in the computing device 180.

In various embodiments, a loading tray 114 may include two or more devices and/or device cases and the test station 160 may test each of the devices in the tray sequentially or simultaneously. After all of the devices are tested, the devices may be sorted, such as with a robotic arm 116.

After a device and/or device case has been cleaned and tested, if it received a grade associated with passing, the device and/or device case may be moved to an offloading station 170 or if it received a grade associated with failing or rejection of the device and/or device case may be moved to a reject station 145. In some embodiments, the index table 110B may rotate (e.g., clockwise relative to the orientation depicted in FIG. 1A) between the stations.

Offloading station 170 may include one or more sorting areas. In various embodiments, one or more sorting areas may be associated with different grades. The offloading station may use the robotic arm 116 to move a device into the one or more sorting areas.

In various embodiments with earbuds, the robotic arm 116 may move one or more earbuds into their respective case, including opening and/or closing the device case. The robotic arm 116 may then move the case containing the earbuds to one or more portions of the offloading station 170.

Additionally, or alternatively, the offloading station 170 may include one or more conveyors 172 to direct the offloaded device and/or device case to where it may be further sorted or packaged for downstream processing. In various embodiments, once devices are graded then the devices may be bulk packaged together for shipment to a retailer or re-packager. In various embodiments, bulk packaging may include directing devices and/or device cases to an autobagger or the like. In some embodiments, the computing device 180 may link one or more of the devices to a recipient and direct the conveyors 172 and/or other sorting and packaging equipment to transmit the device(s) to the recipient, and may be configured to label and/or guide the device(s) for transmission to the recipient.

Reject station 145 may include one or more sorting areas. In various embodiments, each area may be associated with a chute that may direct a device and/or a device case to a portion of the rejection station 145. For example, there may be a portion of the station for each type of failure that a device and/or device case may be found to have. For example, a device may be found to be fraudulent if a device identifier fails to pass an authenticity check, which may be associated with a first portion of the reject station 145. For example, the authenticity check may comprise comparing the identifying information (e.g., serial and/or model number) captured by the optical reader 142 to determine if the device is fraudulent (e.g., no corresponding serial number) or stolen (e.g., on a known list of stolen devices). As another example, a device may fail pairing, which may be associated with a second portion of the reject stations. As another example, a device may fail an audio test, which may be associated with a third portion of a reject station 145. As another example, the reject station may be sorted based on grades, with a first grade associated with a rejection associated with a fourth portion of a reject station 145, a second grade associated with a rejection associated with a fifth portion of a rejection station 145, and the like. In various embodiments, the robotic arm 116 may be utilized to move a device and/or device case to the rejection station 145 and/or a portion of the reject station 145. Alternatively, or additionally, one or more conveyors or other manipulators may be used to move a device and/or a device case to one or more portions of a reject station 145.

The computing device 180 may be a single computer that controls the cleaning and/or testing system 100 or it may be a collection of computers, PLCs, or the like that collectively control the cleaning and testing system 100, whether installed locally with the system and/or remotely positioned and connected via one or more wireless and/or wired communications networks. In some embodiments, each station may also include its own computing device, which may be connected and/or coupled to a central computing device 180 to control and monitor the cleaning and testing system 100.

FIG. 1B illustrates an embodiment of an example cleaning and testing system utilizing a fixture in accordance with the present disclosure. The embodiment of FIG. 1B is a non-limiting illustrative embodiment of the cleaning and testing system 100 described herein and, thus, may include one or more stations, components, fixtures, or the like also described herein. In the embodiment of FIG. 1B, the cleaning and testing station 100 includes a first index table 110A, a second index table 110B, a loading station 120, a pairing station 130, an optical reader 142, a robotic arm 116, a reject station 145, a cleaning station 150, a testing station 160, a conveyor 172, and a computing device 182. FIG. 1B illustrates an embodiment where the first index table 110 includes 8 charging cradles 112A-H and where the second index table includes 4 loading trays 114A-D. Additionally, FIG. 1B illustrates how an operator 190 may be involved to load the charging cradles 112 of the first index table 110A from a loading station 120.

FIG. 2 illustrates an example of a charging cradle 200 in accordance with some embodiments of the present disclosure. The charging cradle 200 may be an embodiment of a charging cradle 112 described herein with respect to FIG. 1A. For example, in some embodiments, the charging cradle 200 may be configured to be installed on the index table (e.g., index table 110A shown in FIG. 1A) or other handling platform. The charging cradle 200 may be sized to have one or more devices or cases containing device loaded onto the charging cradle 200.

In some embodiments, the charging cradle 200 may include a plurality of charging slots 220. In an example embodiment, the device may be one or more earbuds of a pair of earbuds with or without a charging case. The charging cradle 210 may include circuitry needed to charge one or more devices or cases containing device(s) that are placed in the charging slots 220. The charging cradle 200 may be wired or wireless connected to the cleaning and testing system 100, such as to an index table 110. In some embodiments, each slot 220 of the cradle may have a corresponding electrical connector (e.g., USB-C, Lightning connector, or the like) for charging. In some embodiments, one or more wireless charging pads (e.g., Qi charging pads) may be integrated with the cradle for wirelessly charging the device(s). In the embodiment illustrated in FIG. 2, there are four charging slots 220A, 220B, 220C, and 220D. Each of the charging slots 220 may be sized to accept an earbud case comprising a battery. In some embodiments, the charging cradle 210 may be manufactured in multiple SKUs and swappable on the index table depending on the particular device being tested and cleaned (e.g., to facilitate quick retooling). For example, in an embodiment wherein the earbuds are AirPods®, the charging slots 220 may be shaped to each hold an AirPod® case and to charge the AirPod® case either with a wired or wireless connection. As depicted, there are four charging slots 220 allowing for four AirPod® cases to be placed in the illustrated charging cradle 200. Thus the illustrated charging cradle 200 may be used to charge eight AirPods® and four cases when fully loaded. In various other embodiments, there may be a different number of charging slots 220 in one or more rows.

In alternative embodiments, the device may be a watch (e.g., Apple Watch®), the charging slot(s) 220 may correspond to a size of a watch. The charging slot 220 may comprise a protrusion, indentation, or aperture in the charging cradle 200 to allow the device or device case to be placed in the charging cradle 200, including in the charging slot 220, such that the device may be charged.

Once a device is loaded into a charging slot 220, the device may begin to be charged, which may be needed in order for the device to be paired with the cleaning and testing system 100. The device may be charged to a partial charge or the device may be fully charged. In some embodiments, a device may not be paired with the cleaning and testing system 100.

FIG. 3 illustrates an example of a loading tray 300 in accordance with some embodiments of the present disclosure. The loading tray 300 may be an embodiment of the loading tray 114 described herein. The loading tray 300 may include one or more case slots 320 and one or more earbud slots 330. In the embodiment illustrated in FIG. 3, the loading tray 300 includes four case slots 320A-D and four earbud slots 330A-H. In some example embodiments, once a partial charge is imparted to the earbuds and the charging case via the charging cradle, the earbuds may be separated from the case and held separately by the loading tray, so that one or more of the devices may be tested separately. For example, the robotic arm 116 may separate and place the individual ear buds and case in separate slots 320, 330 when transferring between the scanning station 140 of the first index table 110A and the corresponding location of the second index table 110B in the depicted embodiment. Various embodiments of the loading tray 300 may include different numbers of the case slots 320 and earbud slots 330 or dedicated slots for one or more other device types. Each earbud slots 330 may include a fixture or a portion of a fixture as further described herein. Similar to the charging cradle, the trays 114 may have different SKUs and may be swappable for quick retooling to handle different devices. Similarly the computing system 180 and robotic arm 116 may comprise computer program instructions for handling a plurality of device types.

In some embodiments, the loading tray 300 may also include one or more ports that may be connected to by one or more stations. For example, one or more ports may be connected to by a cleaning station 150 to provide a fluid and/or air to a fixture. In another example, one or more ports may be connected to a testing station, such as one or more electrical connections. The connections to a loading tray 300 may include quick connections. In various embodiments, quick connections may be made between a pump and/or a compressor and a fixture and/or loading tray 114.

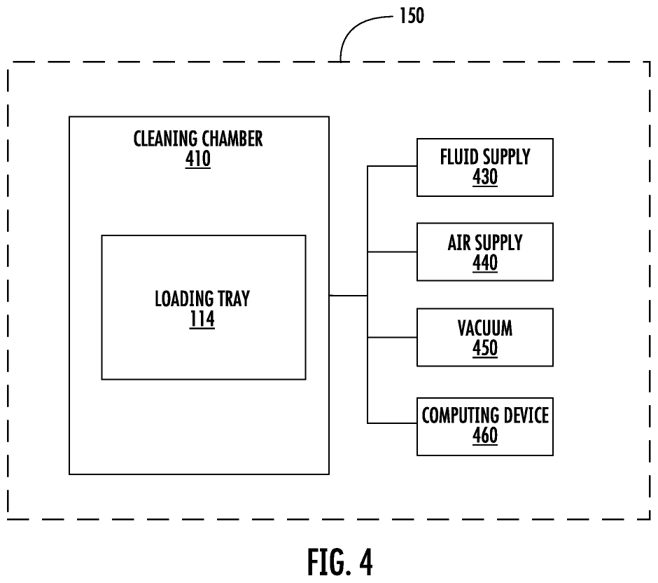

FIG. 4 illustrates an example block diagram of a cleaning station 150 in accordance with some embodiments of the present disclosure. The cleaning station 150 is an example embodiments of a cleaning station 150. The cleaning station 150 may include a cleaning chamber 410, a fluid supply 430, an air supply 440, a vacuum 450, and a computing device 460. The computing device 460 may be an embodiment of the computing device 180 described herein. One or more operations of a cleaning station 150 are described further herein.

A cleaning chamber 410 may be an open or closed chamber that may be used to contain, direct, and/or constrain one or more cleaning fluids and/or gasses relative to the device(s). For examples, in various embodiments the cleaning chamber 410 may include a closed chamber that once loaded may prevent cleaning fluid from leaving the chamber. In this manner, the cleaning chamber 410 may prevents spills or leaks. In some embodiments, the cleaning chamber may be a closed or open area, including a fixed table or portion of the index table, in/on which a sealed fixture may be disposed for directing cleaning fluids and/or gasses within the fixture (e.g., the fixture holding the device may be sealed without requiring a larger chamber).

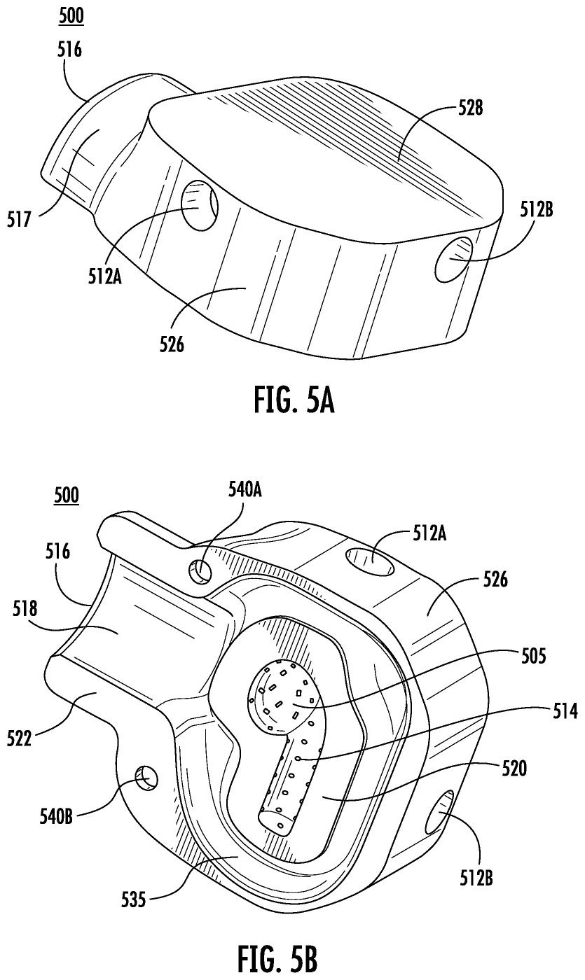

A cleaning chamber 410 may include one or more openings, such as a drawer, side, door, lid, and/or wall that may open and close to allow for the loading and unloading of one or more devices and/or device cases. In some embodiments, the lid of the cleaning chamber may be lowered onto the index table or a sealing surface attached to the index table to enclose the fixture. The drawer, side, door, lid, and/or wall may be coupled to or operated by an actuator that may cause it to open and close. In some embodiments, the chamber structure itself may be moved into position over or adjacent the device(s) and/or device cases. The movement into position may be from the movement of an index table 110B, such as rotating a loading tray 114 into position. A device and/or a device case may be loaded and unloaded as part of loading and unloading of a tray containing multiple devices and/or device cases. Once a tray has been loaded, one or more actuators may cause a drawer, side, door, lid, and/or wall to close, which may seal the cleaning chamber 410 with a watertight and/or airtight seal. In some embodiments, a device and/or device case may be contained in or on a fixture and/or fixture bodies. For example, a robotic arm 116 may remove the device(s) and/or device cases from the loading tray 114 and place them into a dedicated cleaning fixture (e.g., fixture 500, 600, 700 shown in FIGS. 5A-7). In some embodiments, the loading tray 114 may be or may include a dedicated cleaning fixture that moves with the device(s) and/or cases throughout at least a portion of the other steps of the testing and cleaning process. For example, a portion of the loading tray 114/fixture may be mounted to the index table with holes or apertures in the table to deliver cleaning fluid and/or gasses to the fixture, and as the index table rotates the holes or apertures may come into alignment with the supply conduits for such cleaning fluid and/or gasses to automatically connect the fixture to the supplies for cleaning. The supply conduits may be fluidically connected to quick connects terminations and/or injection nozzles, which may be inserted through a hole or aperture in the index table and into external input ports and output ports of a fixture to introduce and/or remove fluid and/or air as described herein.

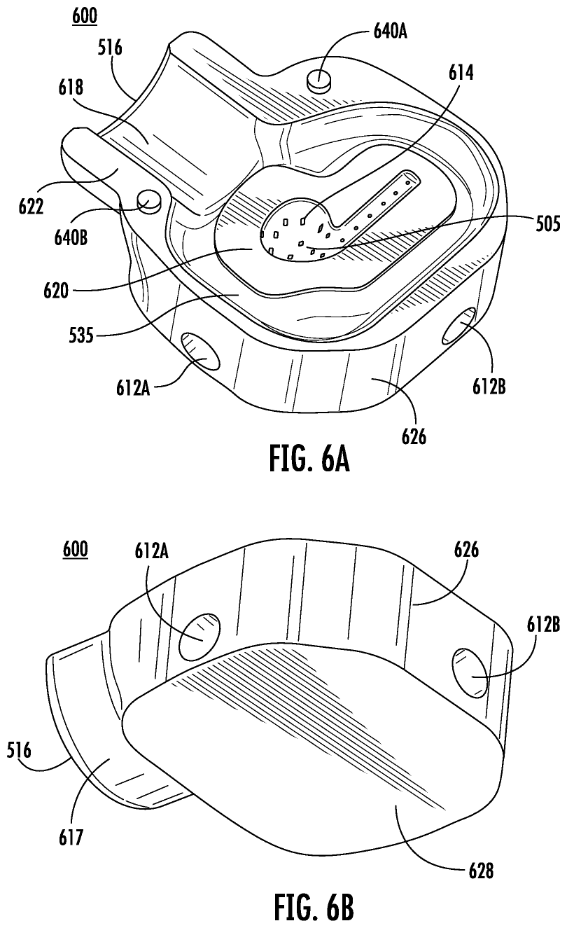

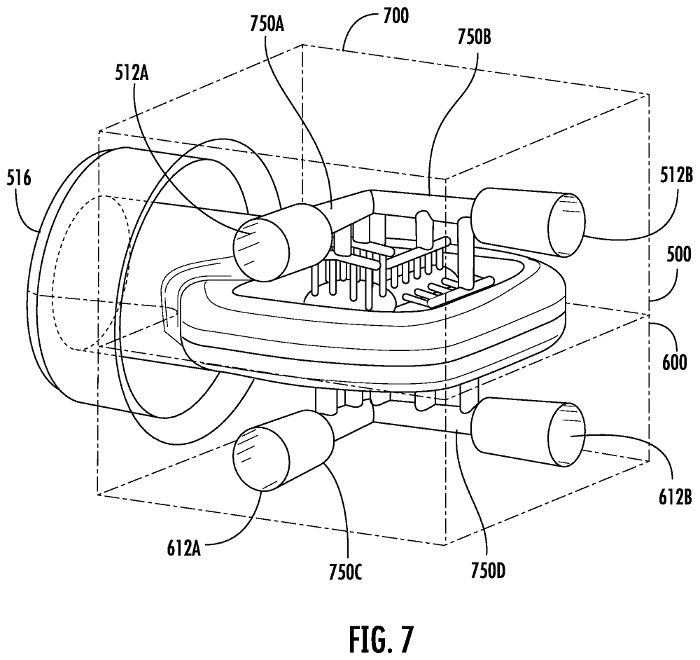

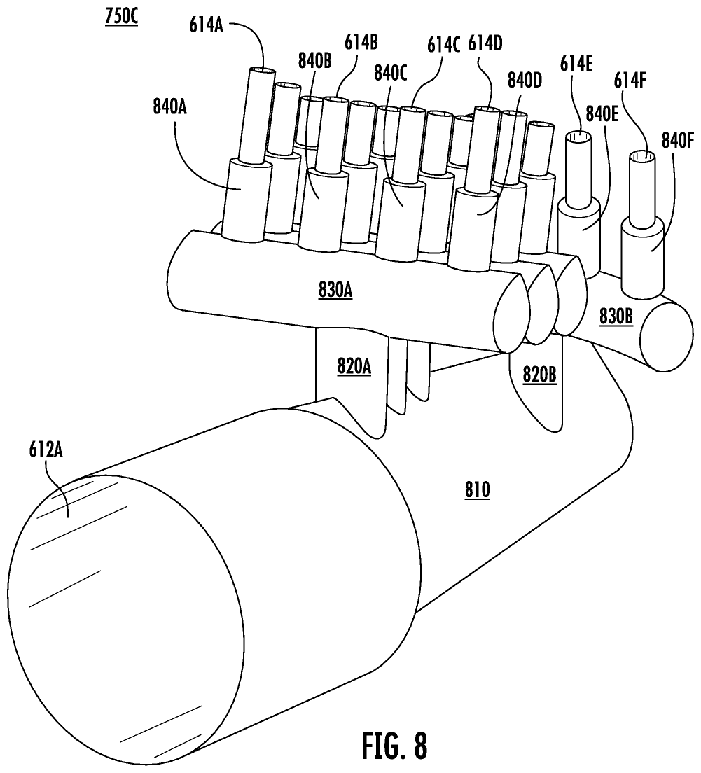

In various examples, a device may have been loaded into a first fixture body (e.g., first fixture body 600 shown in FIGS. 6A-7) and a second fixture body (e.g., first fixture body 500 shown in FIGS. 5A, 5B, and 7) would be mated to the first fixture body to form a full, closed fixture for cleaning, which is described further herein. For example, a lower fixture body may be attached to the rotary index table and may rotate into the cleaning station on the table. An upper fixture body may be disposed at the cleaning station, and an actuator may cause a downward motion of the upper fixture body onto the lower fixture body after the lower fixture body is rotated into position to seal the fixture. In some embodiments, the system may include fewer upper fixture bodies than lower fixture bodies because the upper fixture body(ies) may be reused for each cleaning while the lower fixture bodies may continue with the device(s) and/or device case(s) with the index table. For example, in some embodiments, the system may have more lower body fixtures by a multiple equal to the number of stations on the index table. With the first fixture body mated to the second fixture body then the fixture may be connected to a fluid supply 430 and/or an air supply 440. The mating of the second fixture body to the first fixture body may occur due to the second fixture body being fluidically connected to one or more portions of the cleaning chamber 410 that may be positioned onto the first fixture body. For example, the first fixture body may be seated in a loading tray or on the index table itself, and a robotic arm 116 may position the second fixture body to mate with the first fixture body. Alternatively, the second fixture body may be attached to a moveable portion of the cleaning station 150 that may be positioned or lowered onto the first fixture body. In various embodiments, an operator may position the second fixture body to mate with the first fixture body. In various embodiments herein, cleaning fluid and/or air may be used to clean the device(s). In some embodiments, a vacuum 450 or other negative pressure device may also be connected to the fixture to draw the debris and/or cleaning fluid from the fixture.

In various embodiments, a loading tray 114 may be used. A loading try may contain a number of fixtures, devices (which may be disposed in a fixture or outside of a fixture), and/or device cases (which may also be disposed in a fixture of outside of a fixture). For example, in various embodiments of cleaning and testing earbuds, a loading tray 114 may have places for four sets of earbuds as well as four associated earbud cases. In various embodiments, there may be more or less sets of earbuds and/or associated cases. The earbuds and the associated case(s) may be washed to remove debris, such as earwax, which may be present in both the earbuds and the case(s). In various embodiments, a loading tray 114 may include fixtures for a first device and fixtures for a second device, wherein the first device and the second device are different. For example, the first device may be a first type or model of earbud while the second device is a second type or model of earbud. As another example, the first device may be an earbud while the second device may be a watch, smart phone, mobile computer, tablet computer, wearables, hearables, handheld electronic devices, and the like.

The connection to a fluid supply 430 and/or an air supply 440 may be via one or more quick connects. Quick connects may be connected to a fixture automatically via the cleaning station 140, such as once a loading tray 114 is loaded to cleaning station 150 or a cleaning chamber 410.

In various embodiments including a loading tray 114, the connections of a fixture to the fluid supply 430 and/or air supply 440 may be via the loading tray 114, which may include one or more quick connects. The loading try 114 may route one or more of fluid and/or air through the loading try to one or more input ports of a fixture. Additionally, or alternatively, a loading tray 114 may include one or more connections to an output port of one or more fixtures. In various embodiments, the introduction of fluid into a fixture and allow a fluid to exit the fixture via the loading tray 114. For example, in some embodiments, fixed conduits may be disposed beneath the index table with openings facing upward. The index table, fixture, and/or tray may have corresponding openings that align with the fixed conduits to connect the cleaning fluid, air, and/or vacuum to the respective tray and/or fixture for cleaning the device(s) and case(s) at the cleaning station 150.

In various embodiments, a fixture may be provided for one or more cases. The fixture for a case may be connected to a fluid supply 430 and/or an air supply 440. Alternatively, or additionally, a case may be loaded into a cleaning chamber 410 either in a fixture or outside of a fixture, such as on a loading tray 114. In some embodiments, the case may be cleaned in addition to the device(s) associated with the case. The device case may be cleaned in a same or similar manner as the device, such as with operations and/or functions described herein, which may also include variations to open and/or close a device case. In various embodiments, a cleaning station 150 may clean a device case once in an open configuration and repeat a cleaning a second time in a closed configured (or closed and then open), and a robotic arm 116 may open and/or close the device case between cleanings. Alternatively, in various embodiments where a case may be closed by application of pressure from the injection of one or more fluids and/or air (e.g., a hinged case that may close if pressure is applied to a lid in a first direction), the case may be closed as a part of the cleaning process by application of one or more fluids and/or air in one or more locations in a first direction to cause the device case to close.

Once one or more devices and/or device cases have been loaded to a cleaning chamber 410 then one or more cleaning operations may be performed, such as operations described further herein. The cleaning operations may include cleaning the device and/or device case with a fluid, such as a cleaning fluid (e.g., an ethyl alcohol based fluid), and then allowing the fluid to be removed, such as by exiting a fixture via a vacuum, gravity, or the like. The removal of fluid may be assisted with air from the air supply, such as via positive pressure. Alternatively, the fluid may exit via negative pressure, such as by a vacuum being created, which may extract the fluid from the cleaning chamber 410, fixture, and/or loading tray 114. The application and exiting of fluid may be cycles or iterated, including for one or more periods of time. The periods of time may vary in duration. In various embodiments, the pressure applied and/or vacuum used may be varied, such as using increasing amounts of pressure and/or vacuum.

A fluid supply 430 may include one or more fluid reservoirs, one or more fluid pumps, and/or one or more fluid valves to direct and control fluid from the fluid reservoirs. In some embodiments, the fluid reservoir may be pre-pressurized and/or the fluid supply may be gravity fed. The fluid reservoir(s) may include one or more fluids and/or cleaning fluids. For example, a first fluid reservoir may contain ethyl alcohol. As another example, a second fluid reservoir may contain water. The cleaning station 150, including the computing device 460, may control the one or more pumps and/or one or more fluid vales to provide a fluid from the one or more fluid reservoirs. The providing of these fluids may include mixing the fluids from the one or more reservoirs by controlling the one or more pumps and/or one or more fluid valves, such as using a percentage of a first fluid (e.g., ethyl alcohol) and a percentage of a second fluid (e.g., water).

An air supply 440 may include one or more air compressors, one or more air containers, one or more regulators, and/or one or more air valves. The air supply 440 may provide air to the cleaning station 440, including to a fixture in the cleaning station 150. The cleaning station 150, including the computing device 460, may control the one or more air compressors to provide air, including to provide air to a fixture and/or a fluid reservoir or supply line for driving the fluid. Alternatively, or additionally, air compressor(s) may be controlled to provide pressurized air to one or more air containers, including to provide pressurized air whenever a pressure falls below a threshold. The air supply 440 may use a regulator to regulate the air pressor in the air supply 440. The pressure may be regulated to keep the pressure above a pressure threshold. An example of such a pressure threshold may be 90 psi. In various embodiments, the cleaning station 150 and/or one or more components of the cleaning station 150 may be explosion proof. For example, the air compressor may include an explosion proof motor and the air supply may be rated as explosion proof. Pressurized air in a container may be provided to a fixture by the control of one or more air valves. In various embodiments discussed herein, air, cleaning fluid, and/or combinations thereof may be delivered to the fixture for cleaning the device(s). In some embodiments, a cleaning sequence comprising sequential air and cleaning fluid delivery may be delivered for cleaning the device(s).

In various embodiments, one or more fluids in the fluid supply 430 may be placed under pressure. The air pressure to pressurize the fluid(s) may be provided by the air supply system 440, or the fluid supply system 430 may include its own pump(s), compressor(s), valves, and/or regulator(s) associated with pressurizing the one or more fluids. The pressure may be used to cause the fluid(s) to introduce into a fixture in accordance with one or more cleaning operations described herein.

In various embodiments, one or more air compressors of the air supply 440 may also act as a vacuum pump to create a vacuum to cause the removal of a fluid or air from a fixture or other portion of a cleaning station 150. In some embodiments, one or more separate vacuums (e.g., 450) may be used.

In various embodiments, one or more vacuums 450 may generate a vacuum for use by the cleaning station 150. The vacuum may be used to remove fluid and/or debris from a fixture as described herein.

In various embodiments, a fixture may include one or more fixture bodies that, when mated together, form a cleaning cavity. For example, the fixture may include a first fixture body and a second fixture body. For example, a first fixture body may include a top portion of a fixture and a second fixture body may include a bottom portion of a fixture. In an alternative example, a fixture may include three or more fixture bodies. In various embodiments a fixture may include a first fixture body and a second fixture body. A first fixture body is illustrated from different views in FIGS. 5A and 5B and a second fixture body is illustrated from different views in FIGS. 6A and 6B.

FIGS. 5A and 5B illustrate an embodiment of a first fixture body and FIGS. 6A and 6B illustrate an embodiment of a second fixture body, both of which may be mated to form a fixture in accordance with various embodiments of the present disclosure.

In various embodiments, a cleaning fluid and air may be introduced into a fixture through a plurality of external input ports 512, 612. The fluid and air may be introduced via all of the external input ports 512, 612 at once, through one or more of the of the external input ports 512, 612 while not through others, and/or cycles varying which external input ports 512, 612 are used during different time periods.

FIG. 5A illustrates an example first fixture body 500 from a first perspective in accordance with some embodiments of the present disclosure. FIG. 5A illustrates an exterior portion of the first fixture body 500. The first fixture body 500 may, depending on orientation, be a top fixture body which may be mated to a bottom fixture body.

The first fixture body 500 may include one or more external input ports 512 (e.g., 512A, 512B), a first fixture body output port 516, a first fixture body output port external wall 517, a first fixture body external side wall 526, and a first fixture body external wall 528. The first fixture body illustrated in FIG. 5A and FIG. 5B includes a first external input port 512A and a second external input port 512B.

FIG. 5B illustrates the example first fixture body 500 of FIG. 5A from a second perspective in accordance with some embodiments of the present disclosure. FIG. 5B illustrates an interior portion of the first fixture body 500 as well as some exterior portions of the first fixture body 500.

The first fixture body 500 may include at least a portion of a cleaning cavity 505 (e.g., two or more fixture bodies may individually or collectively define the cleaning cavity) and cleaning cavity ports 514, which may be located and defined by an inner cleaning cavity wall 520. In various embodiments, the inner cleaning cavity wall 520 may define the plurality of cleaning cavity ports 514 of the cleaning cavity 505 by the how each of the cleaning cavity ports 514 are directed into the cleaning cavity 505. The cleaning cavity ports 514 may be evenly distributed through the cleaning cavity 505. Alternatively, the cleaning cavity ports 514 may be unevenly distributed through the cleaning cavity 505, such as to target one or more specific areas of a device or device case in the cleaning cavity 505 (e.g., a speaker outlet on an earbud). The cleaning cavity ports 514 may be shaped to allow for and/or control for the introduction of a fluid and/or air at a high pressure, including at a high speed and associated high force, which may allow for the cleaning of the device. The amount of pressure may allow for cleaning of not only debris but also any viruses from the device.

Various embodiments, such as through adjusting the cleaning cavity 505 with its cleaning cavity ports 514, may be configured to remove debris, viruses, and any other macro- or microscopic particles. This may allow for the cleaning and testing system to be configured to scale based on the desired use case, such as to remove only large debris, which may require less robust cleaning process, such as using fewer operations or iteration of operations. Moreover, controlling of the level of sanitation and/or sterilization may allow for improved cleaning times with faster cleaning operations for less sterile cleaning while also allowing for high levels of sterilization when desired. The type and concentration of cleaning mechanism (e.g., fluid type and/or gas) may also affect the amount of cleaning.

The cleaning cavity 505 may be surrounded by the inner cavity cleaning wall 520. When the first fixture body of the embodiment illustrated in FIGS. 5A and 5B is mated with the second fixture body of the embodiment illustrated in FIGS. 6A and 6B, described herein, the cleaning cavity 505 will be formed by the inner cavity cleaning wall 520 and the inner cavity cleaning wall 620 of the respective fixture bodies. The cleaning cavity 505 is configured to hold a device to be cleaned. In various embodiments, the cleaning cavity 505 may position the device and/or device case in a specific orientation in regard to one or more of the cleaning cavity ports 514, 614.

A cleaning cavity 505 may be contoured for the shape of a device. In various embodiments of devices of earbuds, the cleaning cavity may be contoured for the shape of an earbud. For example, the shape of the cleaning cavity may be determined by a 3D scan of the device(s) to be cleaned and manufactured (e.g., via additive manufacturing, molding, or the like) with a predetermined clearance around the device(s). In various embodiments with a first fixture body 500 and a second fixture body 600, when these two fixture bodies are mated together then the cleaning cavity 505 is configured to surround the earbud or other device(s).

In various embodiments, two or more fixtures used by the cleaning and testing system 100 may each have distinct cleaning cavities that are contoured to a respective device, such as a first model of earbud and a second model of earbud. In various embodiments the contour of the cleaning cavity 505 may be changed by removing a first fixture body or an internal sub-set thereof (e.g., a cleaning cavity body portion) and swapping in another fixture body. Each cleaning cavity body may be a portion of a fixture body that includes the cleaning cavity 505 and is swappable between one or more fixtures. Thus a fixture body may be reused to clean more than device and/or device case or replaced to clean different devices(s) and/or case(s). In some embodiments, the same external input ports of the fixture may be used to connect to a fluid supply 430 and/or an air supply 440 while varying the device and/or device case being cleaned.

In various embodiments, a fixture may be customized for the device to be cleaned. By swapping one or more portions of a fixture to change the cleaning cavity 505 the rest of the fixture 500 stays the same while being able to clean different devices. This allows for an improved system that may be used for a plurality of devices with minimal retooling or adjustment. In various embodiments, a robotic arm 116 may be used to adjust the cleaning cavity 505 of one or more fixtures for one or more devices. The cleaning and testing system 100 may, based on a scanned device identifier, determine the cleaning cavity needed for the device and control the robotic arm 116 to configure a fixture for the device, including swapping out one or more cleaning cavity bodies of one or more fixtures.

Referring to FIG. 5B, the first fixture body 500 may include a first fixture body output port internal wall 518, a first fixture body outer wall 522, and one or more guide cavities 540, such as first guide cavity 540A and second guide cavity 540B. In various embodiments, one or more guides 540 may be used to guide the mating of one of more fixture bodies together.

In various embodiments, each of the cleaning cavity ports 514 is fluidically connected to one of the external input ports 512. This fluidic connection allows for fluid or air input into an external input port 512 to be provided to one or more of the cleaning cavity ports 514.

While the first fixture body of FIGS. 5A and 5B includes two external input ports 512 and an output port 516, is contemplated that a fixture body may have different numbers of external input ports and output ports than illustrated.

In various embodiments, and as illustrated in FIG. 5B, the outer cavity 535 may surround the inner cavity 505. In various embodiments, the outer cavity 535 may surround the outer cavity in one plane. Additionally (not illustrated), the outer cavity 535 may surround the outer cavity in more than one plane. In operation, in various embodiments, fluid and/or gas may enter the cleaning cavity 505 via cleaning cavity port(s) 514, 614 and exit the cavity via a gap between inner cavity walls 520, 620 of the respective fixture bodies 500, 600, which allow the fluid and/or gas to pass into the outer cavity 535 and out the output port 516.

FIG. 6A illustrates an example second fixture body 600 from a first perspective in accordance with some embodiments of the present disclosure. FIG. 6A illustrates an interior portion of the second fixture 600. The second fixture body 600 illustrated in FIG. 6A is analogous to the first fixture body 500 illustrated in FIG. 5B, such as including a cleaning cavity 505, one or more external input ports 612 (e.g., 612A, 612B), cleaning cavity ports 614, a second fixture body output port internal wall 618, an inner cavity wall 620, a second fixture body outer wall 622, a second fixture body external side wall 626, an outer cavity 535, and one or more guides 640 (e.g., 640A, 640B). The second fixture body may have a complementary shape to the first fixture body such that the cleaning cavity 505 therebetween is configured to hold the device(s). The second fixture body illustrated in FIG. 6A and FIG. 6B includes a first external input port 612A and a second external input port 612B. In some embodiments, the fixture may include only a single fixture body, and in some embodiments, the fixture may include more than two fixture bodies.

FIG. 6B illustrates an example second fixture body 600 from a second perspective in accordance with some embodiments of the present disclosure. FIG. 6B illustrates an exterior portion of the second fixture body 600. The second fixture body 600 illustrated in FIG. 6B is analogous to the first fixture body 500 illustrated in FIG. 5A. Thus the second fixture body 600 may include one or more external input ports 612 (e.g., 612A, 612B), a second fixture body output port 516, a second fixture body output port external wall 617, a second fixture body external side wall 626, and a second fixture body external wall 628.

When the first fixture body 500 and the second fixture body 600 are mated, an outport 516 is formed from the first fixture body output port external wall 517 and the second fixture body output port external wall 617. This output port 516 may be connected to one or more output connections in a cleaning chamber. The output port 516 may be utilized to remove debris, fluid, and air from the cleaning cavity. In some embodiments, the fixture bodies may be translated and/or rotated into engagement with each other (e.g., by a robotic arm, motor, conveyor, actuator, or other apparatus).

A fixture, which may have a first fixture body 500 mated to a second fixture body 600, may include an inner cavity 505 and may also include an outer cavity 535. The outer cavity 535 may at least partially surround the inner cavity 505. When the first fixture body 500 and the second fixture body 600 are mated the inner cavity wall 520 of the first fixture body 500 and the inner cavity wall 620 of the second fixture body have a gap between them that allows for fluid, air, and debris to pass from the inner cavity 505 to the outer cavity 535 while maintaining the device in the inner cavity 505. The gap may have a larger minimum dimension than the cleaning cavity ports 514, 614 to facilitate easy removal of debris. In some embodiments, the gap may be sufficiently small to prevent the device(s) or removable portions thereof (e.g., earbud car cups) from passing out of the cleaning cavity. This gap may be referred to as an output opening or an inner cavity output opening. The output opening allows for debris, fluid, and air to pass from the inner cavity 505 to the outer cavity 535. There may be one or more clearances between the inner cavity wall 520 and the inner cavity wall 620 that creates the output opening. The outer cavity 535 may be used to allow fluids, air, and debris to be extracted or removed from the output port 516 of the fixture. This may include debris that may be removed from a device placed into the inner cavity 505. There may be one or more clearances between the device and the inner cavity 505 to allow for the device to be held in the inner cavity while debris may be removed. In various embodiments (not illustrated) the inner cavity wall 520 and the inner cavity wall 620 may form one or more channels between the inner cavity 505 and the outer cavity 535. In various embodiments, a vacuum pump may be coupled or connected to the output port 516 and may generate a vacuum that pulls the fluid and air from fixture. The outer cavity 535 may, in some embodiments, surround the cleaning cavity at least along a plane (e.g., a plane between the fixture bodies) such that fluid and/or gas may exit the cleaning cavity in a full circle around the cavity for efficient cleaning.

In various embodiments, the fixture may not include an outer cavity 535 and the output opening may be directly connected to the output port 516.

In various embodiments, one or more guides 640 and guide cavities 540 may be used to guide one or more fixture bodies together. A guide 540 and guide cavity 640 may be configured and/or shaped to only fit in one orientation. This may assist ensuring a proper fit when two or more fixture bodies are engaged and mated to each other to form a fixture.

In various embodiments, when two or more fixture bodies 500, 600 are mated, the outer body wall 522 and the outer body wall 622 create an airtight seal. The airtight seal may allow for cleaning fluids and air to be introduced into the fixture without exiting the fixture except through the output port 516. Thus the output port 516 may be used to allow cleaning fluids and air to exit the fixture. In some embodiments, one or more adhesives, sealants, gaskets, or the like may be used to further improve the seal between the outer body wall 522 and the outer body wall 622.

FIG. 7 illustrates an example of a fixture 700 in accordance with some embodiments of the present disclosure having the fixture bodies 500, 600 mated. FIG. 7 illustrates a fixture that may comprise, for example, a first fixture body and a second fixture body that are each analogous to the first fixture body of FIGS. 5A and 5B and the second fixture body of FIGS. 6A and 6B, respectively. In some embodiments, the fixture 700 may include four external input ports 512A, 512B, 612A, 612B as well as an output port 716. Illustrated in the embodiment of FIG. 7 are four fluid distribution networks 750A, 750B, 750C, and 750D. Each of the fluid distribution networks connects one of the external input ports to one or more of the plurality of cavity ports of FIG. 7. In some embodiments, each input port may connect to one of a fluid supply or a gas supply. For example, in one embodiment, a first input port 512A, 612A of each fixture body may be connected to a cleaning fluid supply and a second input port 512B, 612B of each fixture body may be connected to a gas supply (e.g., air compressor and/or tank). In some embodiments, only fluid or gas may be connected to each input port 512A, 512B, 612A, 612B. In some embodiments, both fluid and gas may be connected to each input port 512A, 512B, 612A, 612B.

In various embodiments, each of the plurality of cleaning cavity ports (e.g., cleaning cavity ports 514, 614 shown in FIGS. 5A-6B) of the fixture 700 is connected to an external input port via only one fluid distribution network. For example, a fluid input via external input port 512A would travel through fluid distribution network 750A to reach one or more cleaning cavity ports. In this manner, a fluid may be input into an eternal input port 512A and be introduced into a cleaning cavity through a known number of ports. Alternatively, in various embodiments more than one external input port may be connected to more than one fluid distribution network, which may be connected to all or a portion of the cleaning cavity ports. As depicted and described herein, the fluid distribution networks may split the incoming flow from an external input port 512A, 512B, 612A, 612B into a plurality of smaller ports at the cleaning cavity. In some embodiments, the fluid distribution networks may be configured to control the direction, flow rate, and/or pressure of the fluid and/or gas entering the cleaning cavity.

FIG. 8 illustrates an example fluid distribution network 800 of a fixture in accordance with some embodiments of the present disclosure. In the illustration of FIG. 8, the network has been simplified to omit the structure of the fixture body itself for case of viewing the portions of the fluid distribution network. Elements having similar structure should be understood to perform similarly even if not separately labeled. The fluid distribution network 800 may include a plurality of chambers and or channels, and chambers may be connected by channels. In the depicted embodiment, the fluid distribution network 800 includes an input from an external input port 800 connected to a primary chamber 810. The depicted primary chamber 800 is connected to a plurality of primary channels 820A, 820B, each of which is shown connected to a secondary chamber 830A, 830B. Each of the secondary chambers 830A, 840B is depicted in connection with a second set of secondary channels. For example, a first secondary chamber 830A is connected to a plurality of secondary channels 840A, 840B, 840C; and a second secondary chamber 830B is connected to another plurality of secondary channels 840D, 840E, and 840F. In the embodiment of FIG. 8, each of the secondary channels 840A, 840B, 840C, 840D, 840E, and 840F terminate in cleaning cavity port 814A, 814B, 814C, 814D, 814E, and 814F, respectively, which correspond to the various cleaning cavity ports disclosed herein. In this way, a fluid input into external input port 812 may be distributed through the fluid distribution network 800 to cleaning cavity port 814A, 814B, 814C, 814D, 814E, and 814F.

While FIG. 8 illustrates the sizes and shapes of the chambers 810, 830 and channels 820, 840, it is readily appreciated that other shapes, numbers of conduits, denominations of channels (e.g., more or fewer branches), and sizes may be used. A chamber 810, 830 may be shaped to increase flow to a channel 820, 840 while reducing drag and/or turbulence, and a channel 820, 840 may be shaped to increase the flow rate of a fluid at its distal end, such as by tapering or narrowing in diameter or cross sectional area of the channel 820, 840 as it reaches its distal end. Additionally, or alternatively, one or more chambers 810, 830 and or channels 820, 840 may be added or omitted. For example, various embodiments may include one channel 820, 840 that divides into multiple channels 820, 840, or there may be one chamber 810, 830 that connects to a plurality of channels 820, 840 terminating in cleaning cavity ports, or there may be one or more additional numbers of chambers 810, 830 and channels 820, 840. Additionally, while the chambers 810, 830 and channels 820, 840 may be illustrated with circular cross sections, the cross section may be configured to accommodate fluid flow and may be of various shapes. For example, various embodiments may include chambers 810, 830 with tapering or narrowing towards connections to channels 820, 840. In various embodiments, tapering or narrowing may increase a fluid pressure and/or air pressure and a velocity of a fluid and/or air at the tapered or narrow portion or end of a channel 820, 840 (e.g., forming one or more jet nozzles). Additionally, a tapered or narrowed distal end of a channel 820, 840 may allow for the distal ends of channels 820, 840 to be located closer together, such as where the distal ends of one or more channels 820, 840 may terminate as one or more of the plurality of cleaning cavity ports 514, 614. The locations of the tapered or narrowed distal ends of channels 820, 840 may allow for the one or more cleaning cavity ports 514, 614 to be located close together. In various embodiments, the plurality of cleaning cavity ports 514, 614 may be uniformly spaced or evenly distributed in the cleaning cavity 505, 605. Alternatively, the plurality of cleaning cavity ports 514, 614 may include one or more groupings of cleaning cavity ports 514, 614 of the plurality of cleaning cavity ports 514, 614 with the groupings focusing each of the groupings focused on or associated with a particular location(s) of a device. This may allow for one or more groupings to be focused on one or more portions of a device that may receive higher amounts of wear and, thus, may be more likely to need cleaning. In various embodiments, the orientation of the distal ends of one or more of the channels 820, 840 may be configured to allow a fluid and/or air exiting the channel 820, 840 perpendicularly to an adjacent surface of the device being cleaned. Alternatively, the orientation of the distal ends of one or more of the channels 820, 840 may be configured to allow a fluid and/or air exiting the channel 820, 840 at an angle with respect to the device being cleaned.

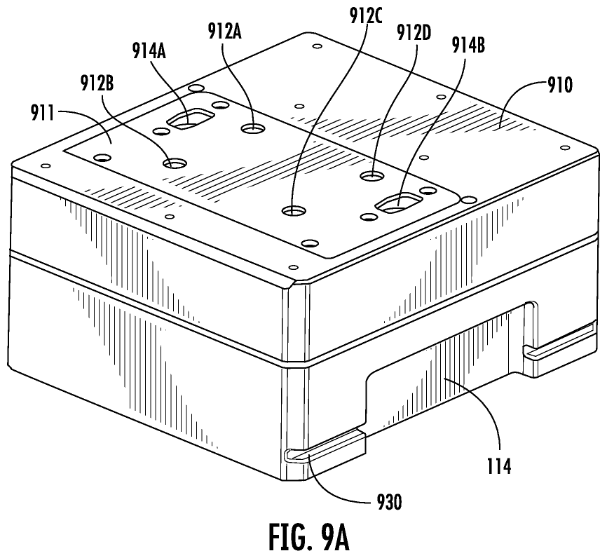

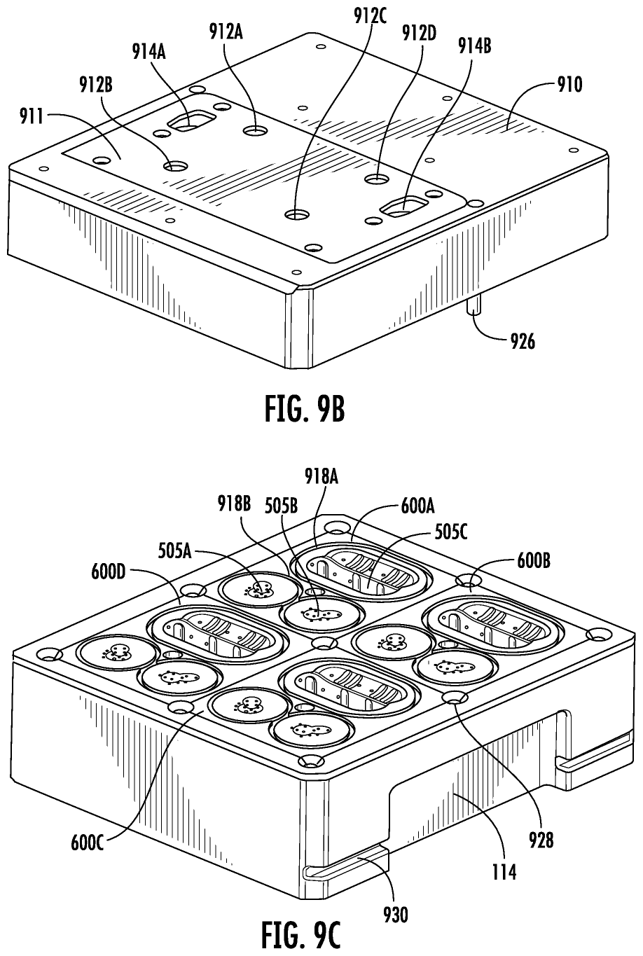

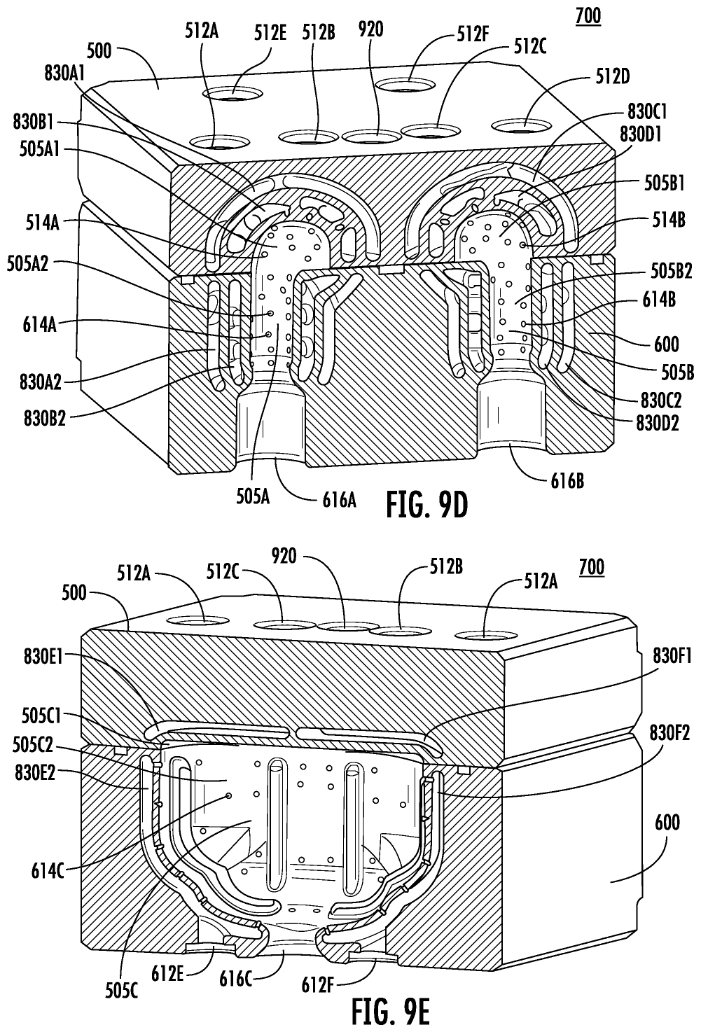

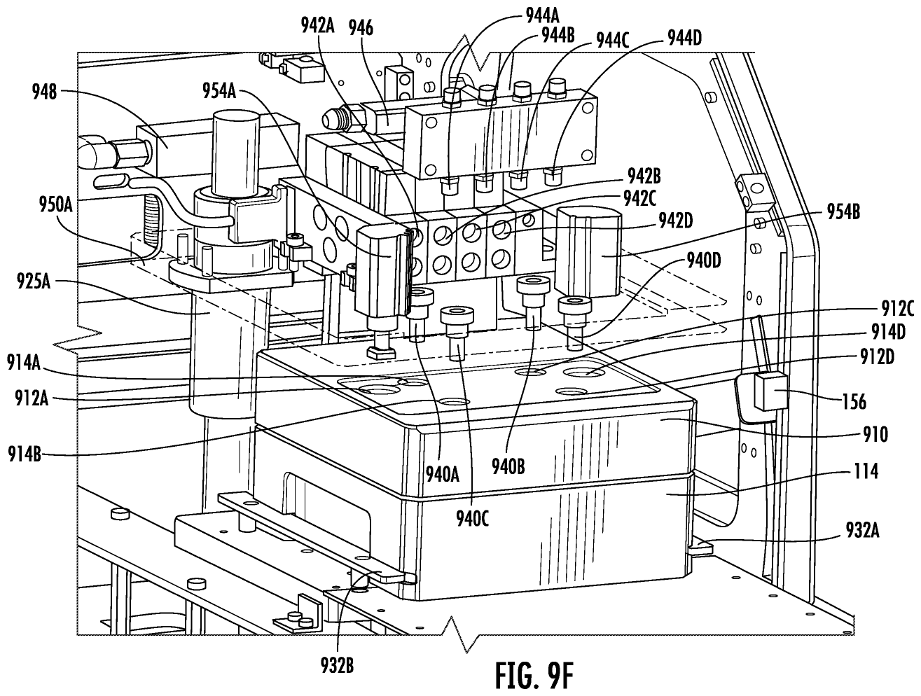

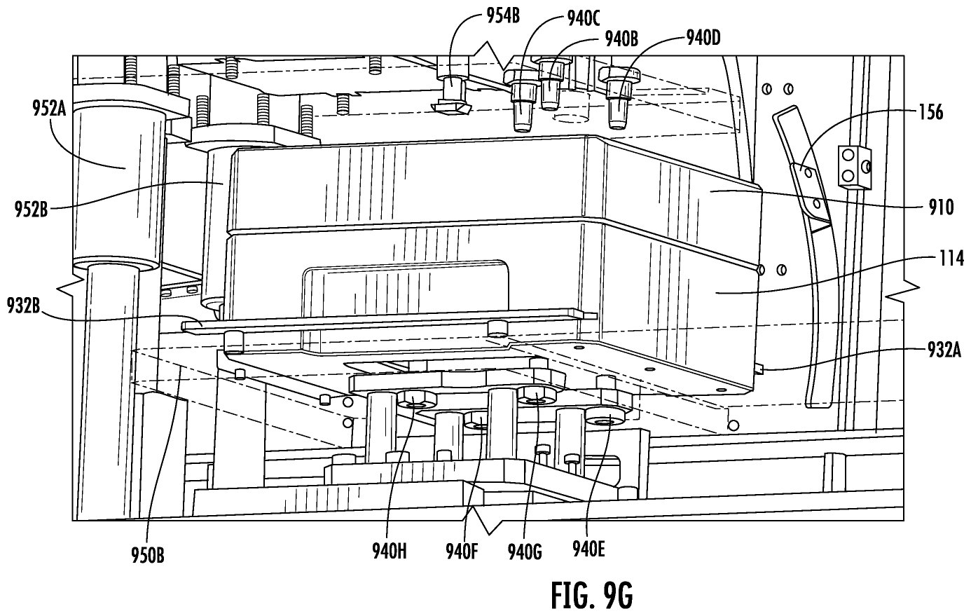

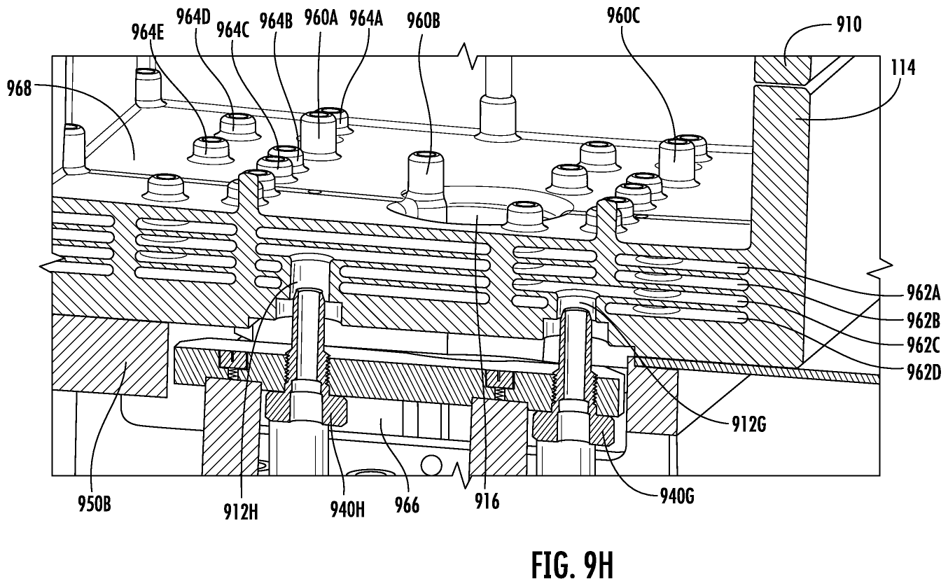

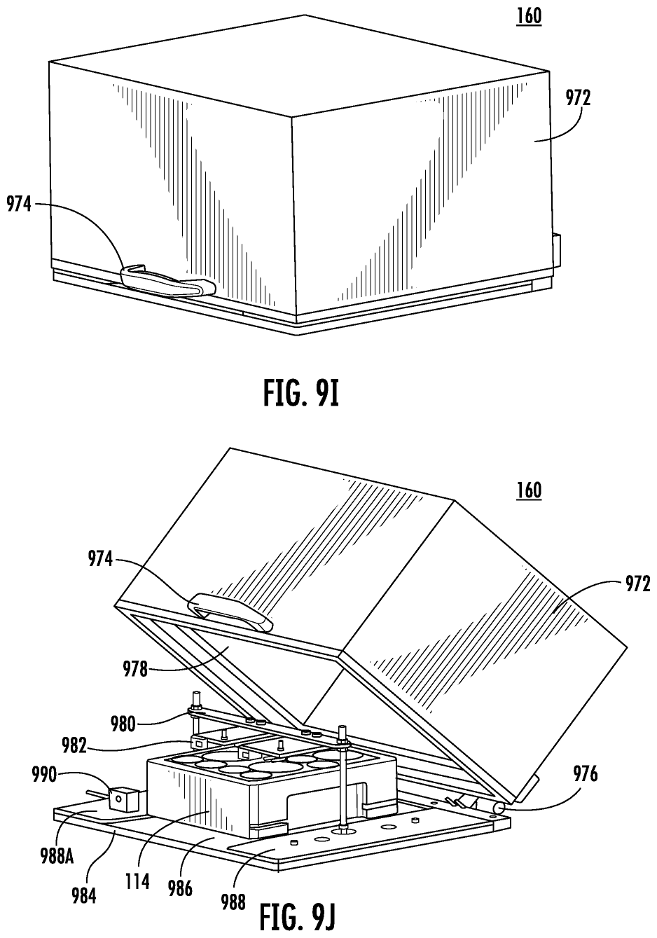

FIG. 9A illustrates an example of a loading tray 114 and a loading tray lid 910 in accordance with some embodiments of the present disclosure. FIG. 9A illustrates a loading tray lid 910 including a plurality of loading tray lid input ports 912 and a plurality of loading try lid locking ports 914. In the embodiment illustrated in FIG. 9A, a loading tray lid plate 911, four loading try lid input ports 912A, 912B, 912C, and 912D, and two loading try lid locking ports 914A and 914B are illustrated.

The loading tray lid plate 911 may be connected and secured to the loading tray lid 910, such as with adhesive, welding, screws, rivets, etc. The loading tray lid plate 911 may include a plurality of openings through which one or more portions of a cleaning station 150 may be inserted, such as quick connects, nozzles, alignment structures, grasping structures, etc. For example a loading tray lid plate 911 may include one or more openings for loading tray lid locking ports 914. The loading tray lid locking ports 914A and 914B illustrated in FIG. 9A are associated with one or more clamps associated with the cleaning station 150. The clamps may be lowered into and through the loading tray lid locking ports 914A and 914B, and then the clamps may be used to secure the loading tray lid 910 and loading tray 114 in position. Additionally, or alternatively, the clamps may be utilized to raise and/or lower the loading tray lid 910 and the loading tray 114.

The loading tray lid input ports 912 may be aligned with or fluidically connected to corresponding input ports 512, 612 of one or more fixtures. In various embodiments, the loading tray lid input ports 912 may pass directly through to the corresponding input ports 512, 612 of one or more fixtures. Alternatively, each of the loading tray lid input ports 912 may be fluidically connected to one or more chambers 962 in the loading tray (see FIG. 9H), and each chamber 962 may be fluidically coupled to one or more corresponding input ports 512, 612 of a fixture. A chamber may be shaped or configured to raise or lower the pressure of a fluid input into the loading tray through a loading tray input port 912. While not illustrated in FIG. 9A, the bottom of the loading tray 114 may include one or more loading tray input ports 912 that may be fluidically connected to a corresponding fixture input port 512, 612 in a similar manner to the top of the tray lid. Additionally, or alternatively, the bottom of the loading tray 114 may include one or more output ports to allow for fluid and/or air input into a loading tray lid 910 and a loading tray 114 to exit (e.g., via vacuum connection or gravity). In some embodiments, the fluid pumped into the fixtures may evaporate in whole or in part.

Such alignment or fluidic connections into and through the loading tray lid 910 and the loading tray 114 allows for a fluid and/or air to be input to a fixture 700 from the cleaning station 150. Similarly, such alignment or fluidic connections also allows for a fluid and/or air to be removed from a fixture 700 from output ports 516 of fixtures 700 through the through the loading tray lid 910 and to the cleaning station 150. Fluid and/or air may be provided from or removed to a cleaning station 150 as described herein, such as with quick connections, which may be inserted into or coupled to loading tray lid input ports 912.

A loading tray 114 may include a loading tray guide cavity 930. The loading tray guide cavity 930 may be shaped and/or configured to match a shape of one or more guides 932 of a cleaning station 150, which may be to align the loading tray 114 when it is placed into a cleaning station 150.

FIG. 9B illustrates an example of a loading tray lid 910 in accordance with some embodiments of the present disclosure. FIG. 9B illustrates a loading tray lid 910, including the loading tray lid input ports 912A, 912B, 912C, and 912D, the loading tray lid locking ports 914A and 914B, and a loading tray lid guide 926. The loading tray lid guide 926 may be a guide to align the loading tray lid 910 with the loading tray 114, such as with one or more loading tray lid guide cavities 928.

FIG. 9C illustrates an example of a loading tray 114 in accordance with some embodiments of the present disclosure. The loading tray 114 may include one or more loading tray lid guide cavities 298. Additionally, FIG. 9C illustrates an example embodiment of a loading tray 114 loaded with four second fixture bodies 600A, 600B, 600C, and 600D. It will readily be appreciated that these four second fixture bodies 600A, 600B, 600C, and 600D engage with four first fixture bodies 500A, 500B, 500C, and 500D, such as described herein. The four first fixture bodies 500A, 500B, 500C, and 500D (not illustrated) may be located in the loading tray lid 910 such that when the loading tray lid 910 is engaged with the loading tray 114 there are four fixtures 700.

As illustrated in FIG. 9C, each fixture 700 includes three cleaning cavities 505A, 505B, and 505C (though it is readily appreciated such cleaning cavities are present for each of the four fixtures while the reference numbers for these cavities 505A, 505B, and 505C are only associated with second fixture body 600A). Each of the fixtures 700, as illustrated, also includes a plurality of gasket cavities 918. As also illustrated, more than one of the cleaning cavities 505A and 505B may share a gasket cavity 918A. Alternatively, a cleaning cavity 505C may have its own gasket cavity 918B. A gasket cavity 918 may be configured to hold, and in operation may hold, a gasket. Such a gasket may further seal a fixture and prevent liquid and/or air from leaking out of a cleaning cavity 505.

FIG. 9D illustrates a first cross-section of an example of a fixture 700 in accordance with some embodiments of the present disclosure. The fixture 700 of FIG. 9D illustrates a cross section of a fixture 700 of a first fixture body 500 (e.g. 500A) and a second fixture body 600 (e.g., 600A), particularly a cross section illustrating cavities 505A and 505B.

The cavity 505A of FIG. 9D may comprise the first cavity portion 505A1 of the first fixture body 500 and the second cavity portion 505A2 of the second fixture body 600. The cavity 505B of FIG. 9D may comprise first cavity portion 505B1 of the first fixture body 500 and the second cavity portion 505B2 of the second fixture body 600.

The first cavity portion 505A1 of the first fixture body 500 includes a plurality of cleaning cavity ports 514A. The second cavity portion 505A2 of the second fixture body 600 includes a plurality of cleaning cavity ports 614A. The first cavity portion 505B1 of the first fixture body 500 includes a plurality of cleaning cavity ports 514B. The second cavity portion 505B2 of the second fixture body 600 includes a plurality of cleaning cavity ports 614B.

As illustrated in the cross section of FIG. 9D, the first fixture body 500 includes a plurality of external input ports 512A, 512B, 512C, 512D, 512E, and 512F. Two of the plurality of external input ports 512A and 512B are associated with the first cavity 505A and two of the plurality of external input ports 512C and 512D are associated with the second cavity 505B. Two of the external input ports 512E and 512F are associated with the third cavity 505C (not illustrated in FIG. 9D).

The first fixture body 500 may also include an alignment port 920, which may be used to align a fixture 700 with one or more protrusions in a loading tray lid 910. The second fixture body 600 may also include one or more alignment ports 920 (not illustrated). Further, an alignment port 920 of the first fixture body may be aligned with an alignment port 920 of the second fixture body to create an opening through the fixture 700 of the first fixture body 500 and the second fixture body 600 so that a protrusion from a loading tray lid 910 or a loading tray 114 may extend through the fixture 700.

As also illustrated in the cross section of FIG. 9D, the second fixture body 600 includes an output port 616A and an output port 616B. The second fixture body 600 also includes a plurality of external input ports not illustrated in this cross section illustration.

The external input ports 512 may be used to introduce fluid and/or air and the output ports 616A, and 616B may be used to remove fluid and/or air from the fixture 700, such as described herein. In the example embodiment illustrated in FIG. 9D there are a plurality of chambers 830, with each chamber associated with an external input port 512.

For the first fixture body 500: external input port 512A is associated with chamber 830A1; external input port 512B is associated with chamber 830B1; external input port 512C is associated with chamber 830C1; and external input port 512D is associated with chamber 830D1. While the external input ports of the second fixture body 600 are not illustrated, for the second fixture body 600: a first external input port is associated with chamber 830A2; a second external input port is associated with chamber 830B2; a third external input port is associated with chamber 830C2; and a fourth external input port is associated with chamber 830D2. Each of these external input ports 512, 612 may provide a fluid and/or air into a fixture body 500, 600, which then enters the associated chamber 830 before being introduced into the associated cavity 505. In various embodiments, the chambers 830 may be isolated from each other (e.g., each chamber 830 feeds separately into the cleaning cavity 505) and may be used sequentially and/or simultaneously during the cleaning operation. In some embodiments, the cleaning cavity ports 514, 614 associated with each chamber 830 may be evenly distributed to provide uniform cleaning from each chamber 830.

FIG. 9E illustrates a second cross-section an example of a fixture 700 in accordance with some embodiments of the present disclosure. The fixture 700 of FIG. 9E illustrates a cross section of a fixture 700 of a first fixture body 500 (e.g. 500A) and a second fixture body 600 (e.g., 600A), particularly a cross section illustrating cavity 505C.

The cavity 505C of FIG. 9E may comprise the first cavity portion 505C1 of the first fixture body 500 and the second cavity portion 505C2 of the second fixture body 600.

The first cavity portion 505C1 of the first fixture body 500 includes a plurality of cleaning cavity ports (not illustrated in this cross-section). The second cavity portion 505C2 of the second fixture body 600 includes a plurality of cleaning cavity ports 614.

As illustrated in the cross section of FIG. 9E, the first fixture body 500 includes a plurality of external input ports 512A, 512B, 512C, and 512D and alignment port 920, which are the same as illustrated in FIG. 9D.

As also illustrated in the cross section of FIG. 9E, the second fixture body 600 includes a plurality of external input ports 612E and 612F and an output port 616C. The plurality of external input ports 612E, 612F may be connected to the plurality of cleaning cavity ports 614, such as with channels 830E2 and 830F2. The second fixture body 600 may also include a plurality of external input ports 612 not illustrated in this cross section (e.g., 612A, 612B, 612C, and 612D, which may be associated with cavities 505A, 505B).

The external input ports 512, 612 may be used to introduce fluid and/or air and the output ports 516, and 616 may be used to remove fluid and/or air from the fixture 700, such as described herein. For the first fixture body 500: external input port 512E is associated with chamber 830E1; and external input port 512F is associated with chamber 830F1. For the second fixture body 600: external input port 612E is associated with chamber 830E2; and external input port 612F is associated with chamber 830F2.

In various embodiments, a chamber 830 may surround a cavity 505 in order to provide a fluid and/or air via a plurality of cleaning cavity ports 514, 614.

In some embodiments, one of the fixture bodies (e.g., the first fixture body) may include a plurality of external input ports 512 to introduce fluid and/or air and another fixture body (e.g., the second fixture body) may include one or more output ports 616 to remove the fluid and/or air. In such embodiments, the fluid and/or air may be introduce through one fixture body and removed through another fixture body. This may include fluid and/or air entering air being introduced through the top of a fixture and having gravity assist in draining the fluid through the bottom of a fixture 700. The cleaning station 150 may also use a vacuum to suction fluid and/or air from a fixture 700 through one or more output ports 516, 616. The fluid and/or air exiting a fixture 700 may exit the fixture 700 into a loading tray lid 910 and/or loading tray 114, which then has one or more output ports for the fluid and/or air to travel through to the cleaning station 150.

In various embodiments, the cleaning station 150 may also include one or more proximity sensors (not illustrated), such at the rear of the cleaning station 150. The one or more proximity sensors may be used to determine that a loading tray 114, including devices and/or device cases in the loading tray 114, have been loaded into the cleaning station 150. In some embodiments, the proximity sensor may be a sensor that may identify one or more protrusions mounted on the loading tray 114. For example, a loading tray 114 may include one or more screws protruding from a side of the loading tray 114, and the proximity sensor may sense the one or more screws, including the type or presence of the metal of the screw, to determine that the loading tray 114 has been loaded. Additionally, or alternatively, the proximity sensor may also determine a specific loading tray 114 (e.g., 114A from 114B), such as based on the one or more protrusions. The specific loading tray 114 may be associated with specific devices and/or device cases, such as from another station in the cleaning and testing system 100.

FIG. 9F illustrates a first perspective of an example of a cleaning station 150 in accordance with some embodiments of the present disclosure. The cleaning station 150, when loaded, may include a loading tray 114 mated to a loading tray lid 910 that hold a plurality of fixtures 700. The loading tray 114 may have been guided into the cleaning station 150 with guides 932A, 932B, which are configured for operation with loading tray guide cavities 930. The loading tray lid may include loading tray lid input ports 912A, 912B, 912C, and 912D and loading tray lid locking ports 914A and 914B.

A cleaning station may include a plurality of surfaces 950, such as a first surface 950A. The first surface 950A may support one or more portions of the cleaning station 150, including actuators, valves, clamps, hosing, connectors (e.g., quick connects and/or nozzles), etc. A first surface 950A may be raised or lowered by one or more lifters 952, such as 952A illustrated in FIG. 9F. The raising or lowering of a surface may cause one or more portions of the cleaning station 150 to be removed from or inserted into a loading tray lid 910 and/or a loading tray 114. The cleaning station 150 of FIG. 9F includes a plurality of connectors 940. A first connector 940A may be associated with loading tray lid input port 912A, a second connector 940B may be associated with loading tray lid input port 912B, a third connector 940C may be associated with loading tray lid input port 912C, and a fourth connector 940D may be associated with loading tray lid input port 912D. Each of the connectors 940 may be inserted into or removed from the associated loading tray lid port 912 by the raising or lowering of the first surface 950A.

A connector 940 may provide fluid and/or air to a fixture 700 via a loading tray input port 912. FIG. 9F does not illustrate hoses or associated portions of the cleaning station 150 fluidically connecting the connectors 940 with other portions of the cleaning station 150, but it will be appreciated that these are present to allow for fluid connections as described herein. For example, a connector 940 may be connected to one or more valves that may be controlled to provide a fluid and/or air at one or more pressures from a fluid supply 430 and/or air supply 440. For example, connector 940A may be connected to a first air supply valve 942A, connector 940B may be connected to a second air supply valve 942B, connector 940C may be connected to a third air supply valve 942C, and connector 940D may also be connected to air supply valve 942D. Each air supply valve 942 may also be connected to air supply 440.

Additionally, connector 940A may be connected to a first fluid supply valve 944A, connector 940B may be connected to a second fluid supply valve 944B, connector 940C may be connected to a third fluid supply valve 944C, and connector 940D may also be connected to fluid supply valve 944D. Each of fluid supply valve 944 may be connected to a fuel rail 946, which may be connected to a fluid supply 430. The fuel rail 946 may include one or more injectors (also referred to as atomizers) configured to mix a liquid and gas stream to atomize one or more fluids (e.g., cleaning fluid) to include in air such that the fluid may introduced, such as in atomized form, into a fixture 700. In various embodiments herein, “fluid” may refer to pure liquid and/or atomized liquid. Each fluid supply valve 944 may also be connected to an air supply 440, which may supply the air that the atomized fluid is injected into. The cleaning station 150 may also include an air regulator 948, a portion of which is illustrated, to an air supply 440.

Additionally, cleaning station 150 may include a lifter 156 that may be connected to a cleaning station lid or cover (not illustrated) that may be raised or lowered to allow for the insertion of a loading tray 114. In various embodiments, and after a loading tray 114 has been loaded into a cleaning station 150, a loading tray lid 910 clamped with one or more clamping actuators 954 may include clamps that may be lowered and/or rotated onto a loading tray 114, such as with the raising or lowering of one or more surfaces 950. A clamping actuator may provide downward pressure or, alternatively, a clamping actuator 954 may be configured with a distal end with one or more projections or shapes configured to be used to be inserted through an opening in the loading tray lid plate 911, rotated, and then pulled up against the bottom of the loading try lid plate 911 to clamp the loading tray lid 910 to the cleaning station 150.

FIG. 9G illustrates a second perspective of an example of a cleaning station 150 in accordance with some embodiments of the present disclosure. FIG. 9G additionally illustrates a second surface 950B, which may support additionally connectors 940 associated with the loading tray 114. Connectors 940E, 940F, 940G, and 940H are illustrated, and these connectors 940 may be raised and or lowered by the second surface 950B, which may be connected to one or more lifters (not illustrated) to raise or lower the second surface 950B. The connectors 940E, 940F, 940G, and 940H may be connected to the air supply valves 940 and fluid supply valves 944, which may provide fluid and/or air to the connector 940 to introduce into a fixture 700 through the loading tray 114.

For example, connector 940E may be connected to a first air supply valve 942A, connector 940F may be connected to a second air supply valve 942B, connector 940G may be connected to a third air supply valve 942C, and connector 940H may also be connected to air supply valve 942D. Additionally, connector 940E may be connected to a first fluid supply valve 944A, connector 940F may be connected to a second fluid supply valve 944B, connector 940G may be connected to a third fluid supply valve 944C, and connector 940H may also be connected to fluid supply valve 944D. In various embodiments, an air supply valve 942 and/or a fluid supply valve 942 may provide air and/or fluid to a connector 940 such that a connector 940 (e.g., 940A) associated with a first fixture body 500 and a connector 940 (e.g., 940E) associated with a second fixture body 600 may each provide air and/or fluid to a cavity simultaneously. In operation, a loading tray 114 and loading tray lid 910 may be translated laterally into the cleaning station 150, and the respective connectors 940 may be raised or lowered into engagement with the assembly to supply the respective fluid and/or gas to the fixture(s) 700.

FIG. 9H illustrates a third perspective of a cross section of an example of a loading tray 114 and loading tray lid 910 in a cleaning station 150 in accordance with some embodiments of the present disclosure. It will be appreciated that the illustration of FIG. 9H omits the fixtures 700 load into a loading tray 114 and loading tray lid 910 for illustrative purposes.

In various embodiments, each of the loading tray 114 and the loading tray lid 910 may include a plurality of protrusions (e.g., 960, 964) protruding into the interior of the loading tray 114 and/or loading tray lid 910. The plurality of protrusions may be protruding from an interior surface 968 of the loading tray 114 and/or an interior surface 968 of the loading tray lid 910.

The plurality of protrusions may include a plurality of alignment protrusions 960. In various embodiments, alignment protrusions 960A, 960B, and 960C may mate to one or more alignment ports 920 of one or more fixtures 700. For example, a first alignment protrusion 9640A may mate to an alignment port 920 of second fixture body 600. Each of the alignment protrusions 960 may include one or more openings, which may allow for a structure (e.g., screw, rod) to extend through the loading tray 114 and/or the loading tray lid 910 and into the fixture 700, which may align the fixture 700, including holding the fixture 700 as well as the loading tray 114 and/or loading tray lid 910 in place.

The plurality of protrusions may also include a plurality of input protrusions 964. The input protrusions 964 (e.g., 964A, 964B, 964C, 964D, and 964E) may mate to one or more external input ports 512, 612 of a first fixture body 500 and/or a second fixture body 600 of a fixture 700. The plurality of input protrusions 964 may be connected, such as with a fluidic connection, to an loading tray input port 912 to introduce a fluid and/or air into a fixture 700, such as via a loading tray chamber 962. In various embodiments, each of the plurality of input protrusions 964 may be fluidically connected to one or more loading tray chambers 962 (e.g., 962A, 962B, 962C, and 962D). It will be appreciated that the loading tray lid 910 may similarly include one or more chambers 962 and input protrusions 964 fluidically connected to introduce a fluid and/or air into a fixture 700.

In various embodiments, each of the plurality of fixtures 700 in a loading tray 114 will be associated with a set of input protrusions 964. Each set of input protrusions 964 for a fixture 700 may include the same number of input protrusions 964. For example, when fluid and/or air is input from a first input protrusion 964 of a first fixture 700 that the same fluid and/or air will be input from a first input protrusion 964 of a second fixture 700. In this manner the device and/or device cases to be cleaned in each of the fixtures 700 may be cleaned simultaneously with the same fluid and/or air. The distribution of fluid and/or air to a first protrusions of each fixture 700 may be by an associated chamber 962, and the same for each second protrusion and third protrusion and so on of each fixture 700. The various chambers may be configured to distribute the fluid and/or air to each of the fixtures (e.g., the air intakes for all fixtures in a tray may connect to the same respective chambers in the tray and the lid), such that actuation of the respective tray and lid supplies of fluid and/or air may simultaneously supply all fixtures.

For example, in various embodiments, connector 940G is mated to loading tray input port 912G, which allows for fluid and/or air introduced from connector 940G to flow into chamber 962C. Chamber 962C may be fluidically connected to one or more fluid input protrusions 964, such as a first fluid input protrusions 964A and a second fluid input protrusions 964B. Thus when fluid and/or air is introduced via connector 940G that fluid and/or air will be introduced into a fixture 700 via the first fluid input protrusion 964A and the second fluid input protrusion 964B. Further, connector 940H may be mated to loading tray input port 912H, which allows for fluid introduced from connector 940H to chamber 962B. Chamber 962B may be fluidically connected to one or more fluid input protrusions 964, such as a fourth fluid input protrusions 964D. It will be appreciated that while the cross section of FIG. 9H may illustrate a chamber 962 as only a portion of a length of the loading tray 114, a chamber 962 may span the length of the loading tray 114.

The loading tray 114 may also include an output port 916. The output port 916 may allow for fluid and/or air to exit the loading tray 114, including to exit from fixtures 700. In various embodiments, the fixtures 700 in the loading tray 114 may, when loaded, leave a gap between the bottom of the fixtures 700 and an interior surface 968 of the loading tray 114. This gap may allow for a fluid and/or air to exit the fixtures and drain from the loading tray 114. The interior surface 968 of the loading tray 114 may be angled or slanted towards the output port 916 to assist the fluid and/or air exiting the loading tray 114. The output port 916 may be connected to one or more additional portions of the cleaning stations via an output connector 966 (e.g., a hose, conduit, etc.), which may direct the fluid and/or air exiting the fixtures 700 and the loading tray 114 out of the cleaning station 150, such as to a drain, to a disposal reservoir, or the like.

FIGS. 9I and 9J illustrate an example of a testing station 160 in accordance with some embodiments of the present disclosure. In the embodiment of FIGS. 9I and 9J the testing station 160 includes an enclosure 972. A handle 974 may be attached to or integrated into the exterior of the enclosure 972. The handle 974 may allow a user or robotic arm 116 to lift the enclosure 972. In some embodiments, the enclosure 972 may be lifted by a lifter, an actuator, or other powered lifting mechanism. The enclosure 972 may be placed on top of a testing station base, which may include a seal and/or gasket for providing sound dampening and/or soundproofing of the enclosure 972. In various embodiments, the testing station base may be connected to an index table, such as index table 110B.

FIG. 9J illustrates a testing station 160 with an enclosure 972 raised, which illustrates that interior of an embodiment of a testing station 160. The enclosure may be opened by, for example, utilizing a handle 974, which may cause the enclosure 972 to open by hinging on one or more hinges 976. In some embodiments, the enclosure may be opened via one or more actuators either automatically or in response to a user actuation. FIG. 9J illustrates one hinge 976, and additional hinges 976 may be located along a first edge of the testing station, such as at a first edge of a testing station base 984. Each of the hinges 976 may be connected to the testing station base 984 and the enclosure 972.

The enclosure 972 may include a cavity in its interior (not numbered), and that cavity may include a testing station lid 978 for the loading tray 114. The testing station lid 978 may be fixedly connected to the enclosure 972 such that when the enclosure 972 is opened then the loading tray lid 978 may be raised. The testing station lid 978 may be configured to fit around a loading tray 114 and riser structures 980. The testing tray lid 978 may include sound proofing material to dampen sound during one or more tests conducted by the testing station 160.

The testing station 160 may include one or more riser structures 980. The riser structures 980 may be connected to one or more sensors 982. In various embodiments, the sensors 982 may include a microphone or a microphone array of a plurality of microphones as described herein. For example, there may be one microphone for each device and/or device case. Alternatively, there may be a microphone array for each device and/or device case. As a further alternative, there may be one microphone or microphone array, and the testing station 160 may be configured to test for a specific response to a test (e.g., audio test) based on the position of a device and/or device case in the loading tray. It will be readily appreciated that additional sensors may be mounted to the riser structure 980 or otherwise supported by the testing station 160 for use during one or more tests conducted by the testing station 160. In operation, the loading tray (e.g., with or without a lid) may be translated into the testing station into a predetermined location relative to the riser structures 980 to ensure consistent testing.

FIG. 9B illustrates a sensor 990. In various embodiments, a testing station will include a sensor 990, which may be a position sensor, a contact sensor, or the like. The sensor 990 may generate a signal when enclosure 972 is closed. The signal from sensor 990 that the enclosure 972 is closed be sent to a computing device 180 that may generate one or more signals that are used to determine that testing may occur or begin. In various embodiments, closing the enclosure 972 may be a quality and/or safety feature to ensure a user's hand or other equipment is not in the enclosure during a test. In various embodiments, the enclosure 972 may close air tight, and closing the enclosure 972 may provide soundproofing or sound deadening for the enclosure 972 to prevent sound from being emitted outside of the testing station 160 and/or prevent sound from entering the testing station 160 during a test.

FIG. 9J further illustrates that the testing station base 984 may include a plurality of base plates 988A, 988B. The base plates 988A, 988B may form a loading tray guide 986. In FIG. 9J the loading tray guide 986 is illustrated as the space between the base plates 988A, 988B that receives and guides the loading tray 114. For example, a user may push or slide the loading tray 114 into the testing station 160 and the loading tray guide 930 allow the loading tray 114 to be positioned in one or more directions in alignment sensor 982. In various embodiments, the base plates 988A, 988B may include one or more raised guides configured to match one or more loading tray guide cavities 930 described herein.

In various embodiments, the testing station 160 may also include one or more proximity sensors (not illustrated), such as mounted to a base plate 988 and/or at the rear of the testing station 160. The one or more proximity sensors may be used to determine that a loading tray 114, including devices and/or device cases in the loading tray 114, have been loaded into the testing station 160. In some embodiments, the proximity sensor may be a sensor that may identify one or more protrusions mounted on the loading tray 114. For example, a loading tray 114 may include one or more screws protruding from a side of the loading tray 114, and the proximity sensor may sense the one or more screws, including the type or presence of the metal of the screw, to determine that the loading tray 114 has been loaded. Additionally, or alternatively, the proximity sensor may also determine a specific loading tray 114 (e.g., 114A from 114B), such as based on the one or more protrusions. The specific loading tray 114 may be associated with specific devices and/or device cases, such as from another station in the cleaning and testing system 100.

Having now generally described several embodiments, various embodiments will now be described in accordance with several example operations.

### Example Operations

In some example embodiments, a cleaning and testing system 100 may perform multiple operations to clean and test a device, including some operations utilizing a fixture 700. Any process description herein including multiple steps will be understood to encompass the full process disclosed as well as any technically feasible subset thereof. FIGS. 9-14 illustrate flowcharts associated with various operations, including operations performed at one or more stations of a cleaning and testing system 100. While the following description includes multiple operations, it is readily appreciated that some of the following operations may be omitted and that additional operations may be included. As is also readily appreciated, some of these operations may be repeated. Additionally, the order of operations should not be interpreted as limiting as the order of these operations may be varied.

The various operations described in the flowcharts herein may use an embodiment with a device of earbuds to provide examples of one or more operations. In embodiments where the device is not an earbud, one or more of the various operations may be omitted or substituted with analogous operations for other devices.

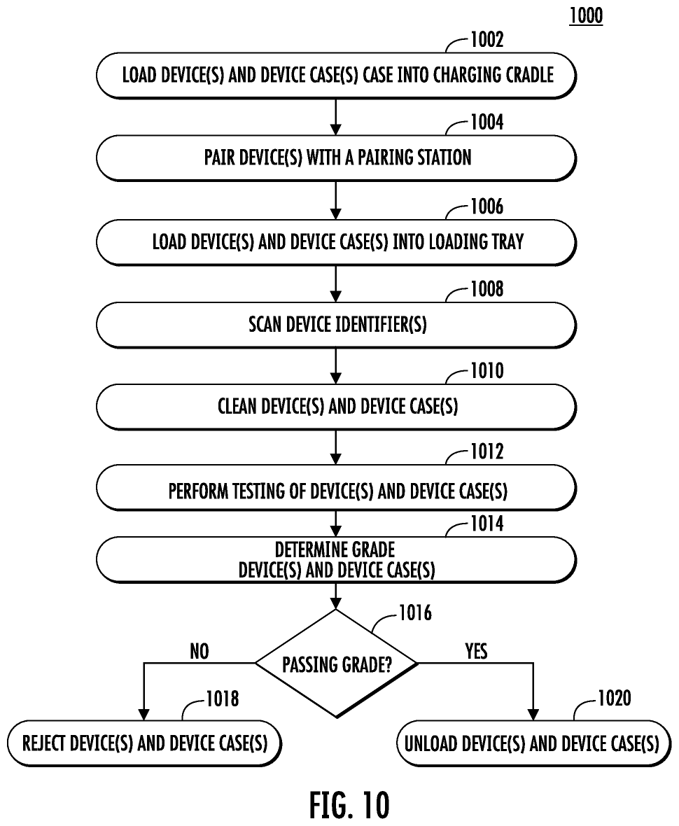

FIG. 10 illustrates an example process 1000 for cleaning and testing a device in accordance with some embodiments of the present disclosure.

At operation 1002, device(s) and/or device case(s) are loaded into a charging cradle 112. They may spend a first period of time in the charging cradle 112, and this first period of time may be the period of time to charge the device and/or associated device case to a minimum level to allow for the completion of one or more other operations described herein. In various embodiments a charge may be provided to a device case which may in turn charge the device. In various embodiments, a device may stay in a charging cradle 112 for a portion for the entirety of the cleaning and testing.

In various embodiments, the charging cradle 112 may be configured to wirelessly charge the device and/or device case. The charging cradle 112 may be connected to one or more power supplies to or of a cleaning and testing system 100, which may allow the charging cradle 112 to provide power the device and/or device case.

At operation 1004, device(s) are paired with a pairing station 130. A pairing station 130 may be used to pair a device and/or device case with the pairing station 130 and/or the cleaning and testing system 100. The pairing station 130 may include one or more actuators that may cause one or more buttons or the like of a device and/or device case to be operated in order to cause the device to pair or become available for pairing. The pairing of a device is additionally described further with respect to FIG. 10.

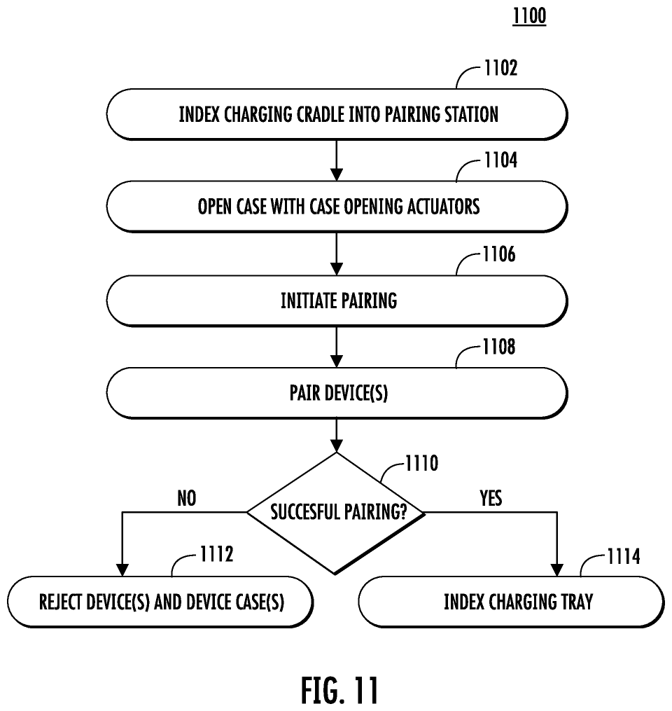

At operation 1006, device(s) and/or device case(s) are loaded into a loading tray 114. A device and/or device case may be loaded from a charging cradle 112 into a loading tray 114. The loading tray 114 may include a fixture or a fixture body for the device and/or device case. In various embodiments, a charging cradle 112 may not be configured to be used with a cleaning station 150 or a testing station 160 while a loading tray 114 may be configured for these additional stations. Loading a device and/or a device case is additionally described with respect to FIG. 11.

At operation 1008, a device identifier(s) are scanned. A device identifier may be located on a device and/or a device case. A device identifier may uniquely identify the device, such as with a serial number, or may contain such information in a coded format, such as a barcode, QR code, or the like. The device identifier may be scanned, such as with an optical reader of a scanning station 140. Scanning a device identifier is further described with respect to FIG. 11.

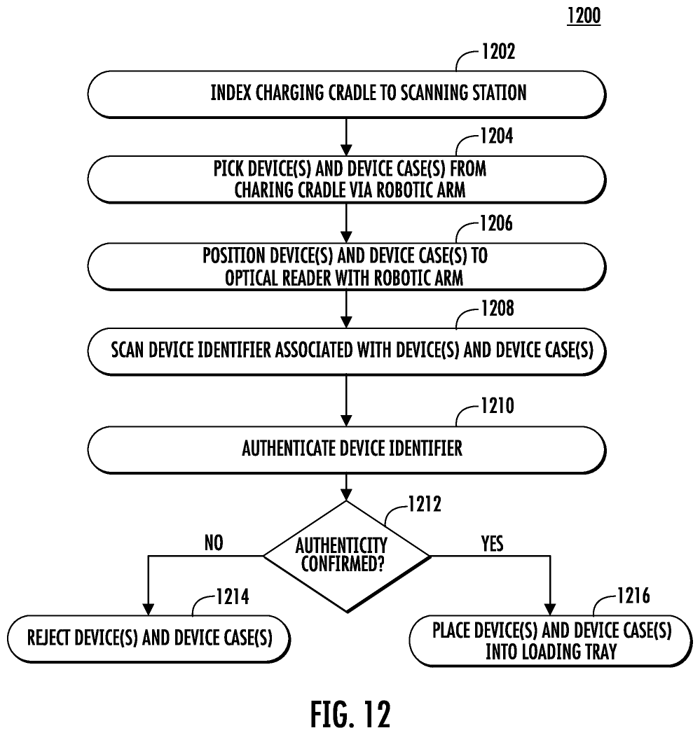

At operation 1010, device(s) and/or device case(s) are cleaned. A cleaning station 150 in accordance with the various embodiments described herein may be used to clean a device and/or a device case. The cleaning station 150 may connect to one or more ports of a loading tray 114 or a fixture containing a device and/or a device case to provide one or more cleaning fluids to clean a device. Cleaning a device and/or a device case is further described with respect to FIG. 12.

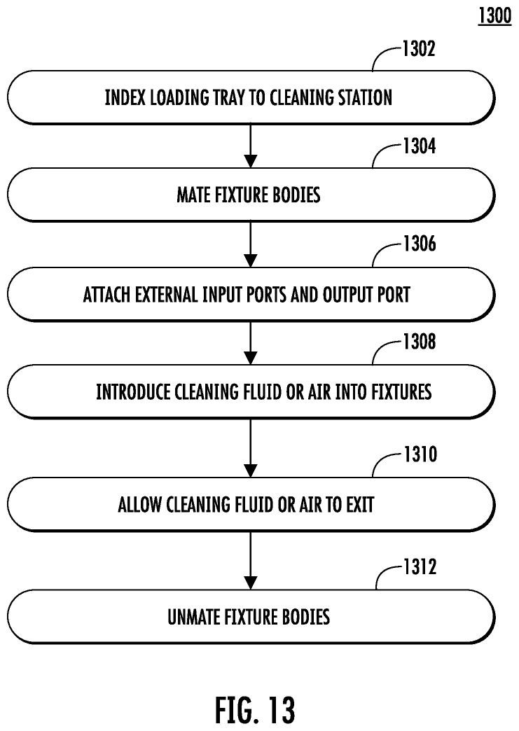

At operation 1012, devices(s) and/or device case(s) are tested. A device and/or device case may be tested with a testing station 160. The testing station 160 may include one or more sensors that may be used to administer one or more tests to determine a response from the device and/or device case. Testing a device and/or a device case is further described with respect to FIG. 13.

At operation 1014, a grade is determined for the device(s) and/or device case(s). Based on, among other things, the pairing, the scanning, the cleaning, and/or the testing, one or more grades may be assigned to the device and/or device case, which may include one or more grades associated with passing a test and one or more grades with failing a test. In some embodiments the grade may indicate, among other things, that the device and/or device case is clean and functional or, alternatively that the device is not functional.

At operation 1016, it is determined if the device(s) and/or device case(s) passed testing or not. In various embodiments, a pass or fail grade may be assigned to a device. Alternatively, a grading scale may be used to assign one of a plurality of grades to a device. A grading scale may use an A, B, or C grading to indicate passing and a D and F scale to indicate failing. The grading scale may be based on how the device performed during testing or the physical condition of the device, such as how worn a device is. The test performance may be generated by the testing station 160. The physical condition of the device may be generated by the scanning station 140.

In various embodiments, a grading scale may be used to determine if and how a product is rejected or unloaded. Various embodiments may include one or more areas of a rejection station 145, which may include different areas for each reason a device and/or case may be rejected. For example, there may be an area of the rejection station 145 for devices that failed to pair, that failed to pass an authenticity check, that failed to pass a test from a testing station 160, etc.

As an additional example, there may be one or more areas of an unloading station 172 that may be designated for different passing grades. In various embodiments one passing grade may be like new, and there may be areas for devices receiving this grade. There may also be an area for gently worn, heavily worn, functional but physically damaged, etc. Each of these areas may be associated with how to unload the device and/or device case from the cleaning and testing system 100. Some grades may be associated with individually packaging an item and other grades may be associated with bulk packaging an item. Similarly, some rejected grades may be associated with various downstream operations. For example, a first example rejection grade may be associated with a faulty but repairable device that is sent to be refurbished. A second example rejection grade may be associated with a faulty and unrepairable device that is sent to be reclaimed for parts. A third example rejection grade may be associated with a completely unsalvageable device that should be discarded or recycled. In some embodiments, as part of the grading process, the computing device 118 may detect and diagnose one or more faults with the device(s) and/or case(s) based on the test results and recommend one or more further remedial steps to further classify and/or resolve the fault.

At operation 1018, if the device(s) and/or device case(s) did not pass testing, the device(s) and/or device case(s) are rejected. Rejected device(s) and/or device case(s) may be moved to a reject station 145, such as with a robotic arm 116 and/or one or more conveyors.

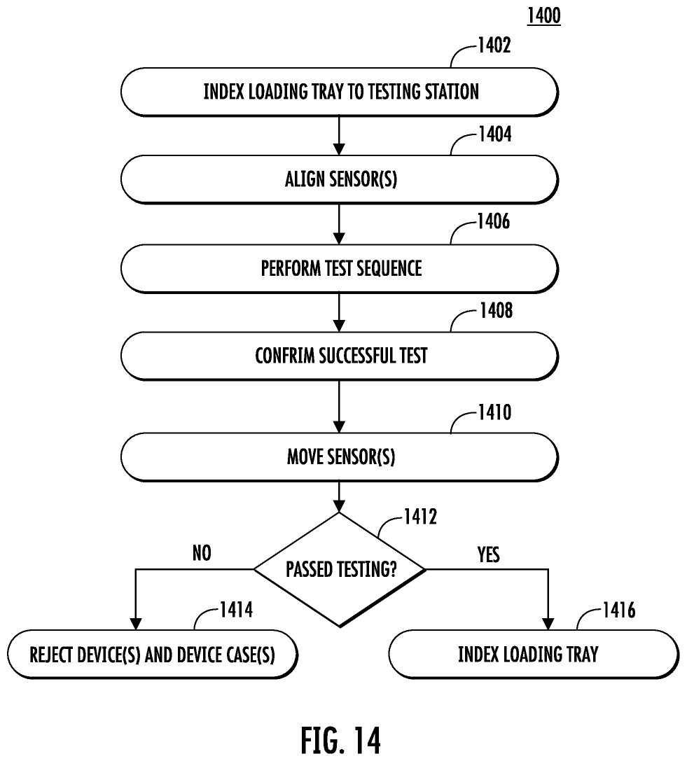

At operation 1020, if the device(s) and/or device case(s) passed testing, the device(s) and/or device case(s) are unloaded. Rejected device(s) and/or device case(s) may be moved to a reject station 145, such as with a robotic arm 116 and/or one or more conveyors. Unloading a device and/or a device case is further described with respect to FIG. 14.

FIG. 11 illustrates an example pairing process 1100 in accordance with some embodiments of the present disclosure.

At operation 1102, a charging cradle is indexed to a pairing station 130. A cleaning and testing system 100 may use charging cradles to place a charge onto a device and/or a device case. The charging cradle, having charged a device and/or a device case to a minimum threshold, may be indexed to a pairing station 130. The minimum threshold may be a charge level or a power level. The charging cradle 112 may include one or more sensors to determine the amount of charge on a device and/or a device case, which may include receiving from the device and/or device case what the charge or power level currently is. Alternatively, a minimum threshold may be associated with a period of time to charge a device and/or a device case to minimum threshold. In various embodiments, the period of time may be controlled by the movement of an index table 110, which may not index the charging cradle 112 to a pairing station until the period of time to charge a device has passed.

At operation 1104, a case is opened with case opening actuators. Once a device and/or a device case is indexed to a pairing station 130, the pairing station may open the case with one or more actuators. Alternatively, or additionally, a robotic arm 116 may be used to operate the case. If a device case is not present, the actuators or robotic arm 116 may be used to manipulate the case, such as positioning the case so that one or more pairing operations may occur.

At operation 1106, pairing is initiated. In various embodiments, pairing may be initiated by making a device and/or a device case available for pairing. This may include using one or more actuators to operate one or more buttons on a device and/or device case.

At operation 1108, a device is paired. To pair a device the pairing station 130 may determine that the device is available for pairing and select to pair with the device.

In various embodiments involving Bluetooth® pairing, a device may be first made available for pairing, a pairing station 130 may identify that the device is available for pairing, the pairing station 130 may send a request to be paired, and the pairing station 130 and the device may be paired in response to the request.

At operation 1110, it is determined if there was a successful pairing. One or more of the operations of 1102 to 1108 may have prevented pairing. If a device is not successfully paired then the cleaning and testing system 100 may proceed to operation 1112. If a device is successfully paired then the cleaning and testing system 100 may proceed to operation 1114.

At operation 1112, if it is determined that there was not a successful pairing, the device and/or device case are rejected. On a determination of an unsuccessful pairing, a device and/or device case may be assigned a grade and may be rejected and moved to a rejection station 145, such as with a robotic arm 116 and/or one or more conveyors.

At operation 1114, if it is determined that there was a successful pairing, the charging tray is indexed. The charging tray 112 may be indexed to another station, such as scanning station 130.

In various embodiments with earbuds as devices, a charging cradle 112 containing a plurality of pairs of earbuds and associated cases may charge the cases and, in turn, the cases may charge the earbuds. The time the earbuds and associated cases spend on the charging cradle 112 may be for a first period of time to charge the earbuds to a minimum level of charge predetermined to be sufficient to allow for cleaning and testing of the earbuds and cases. After a sufficient charge has been applied, a rotary table 110A may index the charging cradle 112 to a pairing station 130. To pair with the earbuds, the associated case may need to be opened. At the pairing station 130, if a case is closed, the pairing station 130 may open the case with an actuator. After the cases are opened the earbuds may be exposed and the pairing may be possible. To make the earbuds available for pairing, which may require a button on an associated case to be operated. The pairing station 130 may operate the button, which may include holding a button depressed for a period of time. Once the earbuds are put into a pairing mode and available for pairing, the pairing station 130 may send a request to pair, which may cause the earbuds to be paired to the pairing station 130. If any of the earbuds and cases are not successfully paired, these earbuds and cases may be moved from the charging cradle 112 to a reject station 145. On successfully pairing of earbuds and associated cases, the earbuds and associated cases may be moved to another station, such as by indexing a charging cradle 112 with the rotary index table 110A.

FIG. 12 illustrates an example scanning and process 1200 in accordance with some embodiments of the present disclosure.

At operation 1202, a charging cradle 112 is indexed to a scanning station 140. The charging cradle may include one or more devices and/or device cases. In various embodiments a device may be indexed to a scanning station 140 without the use of a charging cradle 112.

At operation 1204, device(s) and/or device case(s) are picked via the charging cradle with the robotic arm 116. The robotic arm 116 may be configured with one or more grasping elements (e.g., fingers, magnets, forks, or the like) to grasp or otherwise manipulate a device and/or a device case. The robotic arm 116 may remove the device and/or device case from the charging cradle 112 once grasped.

At operation 1206, device(s) and/or device case(s) are positioned via the charging cradle with the robotic arm 116. The robotic arm 116 may, after removing a device and/or a device case from a charging cradle, move the device and/or device case in view of an optical reader 142. The optical reader may capture an image of the device and/or device case. The captured image may include an image of the device and/or device case generally or from one or more predetermined angles. The capture image may include a device identifier.

At operation 1208, a device identifier associated with the device and/or device case is scanned. Scanning a device identifier may be done with an optical reader 142 capturing an image of a device identifier and decoding the device identifier, which may be done with optical character recognition, barcode decoding, QR code decoding, or the like. Scanning the device identifier allows for the scanning station 140 to determine the information in the device identifier, which may include a serial number, a model number, or the like that uniquely or generally identifies the device and/or device case, which may allow for the device to be authenticated.

At operation 1210, the device identifier is authenticated. In various embodiments, the scanning station 140 and/or the computing device 180 may authenticate a device identifier. Devices may be counterfeited, and the authentication may determine if a device identifier is associated with an authentic device. In some embodiments, the identifying information and/or any other visual information may be captured off the device(s) by the optical reader in addition to or instead of authenticating the device(s).

In various embodiments, the scanning station 140 and/or the computing device 180 may include or be able to access serial numbers or authentication information associated with one or more lists of authentic devices. The serial number determined from a scan of a device identifier may be compared against such lists to determine if a device is authentic. Authenticating a serial number may include determining if a serial number complies with a format, including but not limited to a alphanumeric format, which may also include symbols. In various embodiments, one or more lists of authentic devices may be provided by or made available by a manufacturer. This may include a cleaning and testing system 100 communicating with a manufacturer's device or system to receive a list of authentic devices and/or list of serial numbers. Alternatively, or additionally, a cleaning and testing system 100 may communicate one or more serial numbers or authentication information with a manufacturer's device or system and the manufacturer's device or system may, in response, transmit an indication of authenticity indicating if the one or more serial numbers or authentication information are authentic.

In various embodiments, a device identifier may also be used in conjunction with a previous image of an authentic device to determine if the captured image of the device and/or device case matches or is analogous to the image of the authentic device. Thus a serial number as well as image recognition may be used to authenticate a device. In various embodiments, image recognition may also be used to determine if a serial number or authentication information is located on and/or at a first portion of a device and/or device case associated with an authentic device. If it is determined that a serial number or authentication information is not located in the correct first portion of a device and/or device case, then authenticity may not be confirmed.

In various embodiments, if both the serial number and/or authentication information as well as location of the serial number and/or authenticity information matches those of authentic devices and/or device cases then authentic is confirmed. In some embodiments, the process may include confirming that the serial number and/or authenticity information matches one or more allowed product types (e.g., corresponding to the system and/or fixture in which the cleaning will take place or the intended product for processing).

At operation 1212, it is determined if authenticity was confirmed. If authenticity is not confirmed, the device may be graded and the device and/or device case may be rejected at operation 1214.

If authenticity is confirmed, the device may be graded and the device and/or device case may be placed into a loading tray 114 at operation 1216. Placing the device and/or device case into the loading tray 114 may be done with the robotic arm 116.

In various embodiments, such as those that may not use a charging cradle 112 or a loading tray 114, a rejected device and/or device case may be removed to a reject station 145. The authenticated device may be moved, such as by indexing an index table 110 or by one or more conveyors to another station.

FIG. 13 illustrates an example cleaning process 1300 in accordance with some embodiments of the present disclosure.

At operation 1302, a loading tray 114 is indexed to a cleaning station 150. A loading tray 114 may be moved to a cleaning station 150, such as via an indexing table. Alternatively, a robotic arm 116 may move the loading tray to a cleaning station 150.

At operation 1304, fixture bodies are mated. In some embodiments, the device(s) and/or case(s) may be moved into a bottom fixture body before the fixture bodies are mated. In some embodiments, the device(s) and/or case(s) may already be in the bottom fixture body. The loading tray may include one or more devices and/or device cases loaded into a first fixture body. In various embodiments, it may be at the cleaning station 150 that a device and/or device case may be loaded to a first fixture body. Subsequently one or more second fixture bodies may be mated to the first fixture body containing the device and/or device case to form a fixture. The mating may occur using one or more fixture guides and fixture cavities of the fixture bodies. The mating may be performed by the cleaning station 150, such as by using actuators to place second fixture body, or by a robotic arm 116. In various embodiments, a fixture may be mated and the fixture bodies may be secured using a clamp, latch, actuator arm, actuator of the cleaning station 150, or the like.

At operation 1306, external input ports and output ports are attached. One or more input ports 512, 612 and one or more output ports 516 of a fixture 700 may be connected to the cleaning station 150. The input ports 512, 612 may be connected to a fluid supply 430 and/or an air supply 440. The output port 516 may be connected to a fluid supply 430 and/or an air supply 440. Each of the fluid supply 430 and the air supply 440 may be configured to provide fluid or air, respectively, and also to receive fluid or air, respectively, after it has been used during one or more cleaning operations. In this manner cleaning fluids may be provided and removed from a fixture. The connection of a fluid supply 430 and an air supply 440 may be done with quick connects that may be connected and removed from a fixture 700 by a cleaning station 150, such as with use of one or more actuators or the like. In some embodiments, the quick connects may be automatically connected upon indexing the loading tray to the cleaning station and/or upon the closing or loading of a portion of the cleaning station and/or fixture bodies.

At operation 1308, cleaning fluids or air are introduced into a fixture 700. One or more operations described herein may introduce a cleaning fluid. Introducing a cleaning fluid may include mixing one or more fluids by a cleaning station 150, such a with one or more valves of a fluid supply 430 causing a plurality of fluids to be mixed. The cleaning fluid may then be pumped into a fixture via the connections of the cleaning station 150 to the external input ports 512, 612 of a fixture 700. In various embodiments, the cleaning fluid may be atomized, and the atomized cleaning fluid may be introduced to a fixture 700. In various embodiments, atomizing the fluid prior to being introduced may provide improvements, including lower the quantity of liquid required and allowing the atomized liquid to be introduced at a higher velocity compared to non-atomized liquid.

At operation 1310, cleaning fluids or air are allowed to exit. The cleaning fluids may exit a fixture 700 through an output port 516. A cleaning station 150 may turn off a pump of a fluid supply 430, which may cause the cleaning fluids to exit the fixture 700. In various embodiments, an air supply 440 may include a compressor or pump that may be used to create a vacuum. The vacuum may be applied to the output port 516, which may cause the cleaning fluids to be vacuumed out of the fixture 700. In various embodiments, air supply 440 may include a pressurized container containing air that has been pressurized by a compressor. The compressed air may be introduced into the external input ports 512, 612 and cause the cleaning fluid to exit the fixture due to air being flushed through the fixture.

In various embodiments, the introduction and cleaning fluids and/or air of operation 1308 along with a time period to allow the cleaning fluids and/or air to exit of operation 1310 may be referred as a cycle, and cycles may be iterated. Each iteration may be performed for a one or more periods of times. In various embodiments, each iteration may utilize the same cleaning fluid or a different cleaning fluid, which may be a different mixture of one or more fluids stored in the fluid supply 430. In various embodiments, each cycle may use one or more external input ports 512, 612, and multiple cycles may use different external input ports 512, 612 for each of the cycles. Multiple cycles together may be referred to as a cleaning program or program. A cleaning program may be controlled or executed by a computing device 180 and/or a computing device 460, including controlling or executing one or more operations of the cleaning station 150, including by controlling the fluid supply 430, the air supply 440, and the vacuum 450.

In some embodiments, an additional step of rotating the fixture and running another cycle of fluid and/or gas through the fixture may be performed. For example, rotating the fixture may allow debris to settle out of previously-upturned portions of the earbuds for removal, and may help to dislodge debris and/or void air bubbles that may have formed on the underside of the device(s).

At operation 1312, fixture bodies are unmated. After one or more cleaning cycles have been completed, the fixtures bodies are unmated. Before being unmated, connections to the cleaning system 150 may be removed, such as quick connects. The fixture bodies may be removed from each other in the reverse of how they were mated, which may be performed by the cleaning station 150, by a robotic arm 116 or the like.

In various embodiments, a cleaning station may use multiple cleaning cycles to clean a device in a fixture 700. After a fixture 700 is loaded, a cleaning station 150 use a cycle that applies a cleaning fluid for 20 seconds and then applies air for 20 seconds. The cleaning cycle may be repeated more than once, such as two or three cycles being performed. The cleaning fluid may be a mixture of two or more fluids, such as a mixture of 80% ethyl alcohol and 20% water, which may be mixed by controlling one or more valves of a fluid supply 430 that supplied the cleaning fluid used in the cleaning cycle.

In various embodiments, a cleaning cycle may include introducing a cleaning fluid via all of the external input ports 512, 612 at once or may introduce a cleaning fluid via one or more external input ports 612, 612 of a fixture a time. For example, a cleaning fluid may be introduced via external input port 512A of a fixture 700, then air may be introduced via external input port 512A, then a cleaning fluid may be introduced via external input port 512B, then air may be introduced via external input port 512B, then a cleaning fluid may be introduced via external input port 612A, then air may be introduced via external input port 612A, then a cleaning fluid may be introduced via external input port 612B of a fixture 700, and then air may be introduced via external input port 612B. In such a manner, each external input port 512, 612 may be used to introduce a cleaning fluid and air, which may be done for one or more predetermined periods of time. The period of time may be the same for each external input port 512, 612 or may be different.

In various embodiments, the connection to the external input port 512, 612 may be a single connection that supplies both cleaning fluid and air. This may allow for air being supplied to clean the previously supplied cleaning fluid out of the connection to the fixture 700.

In various embodiments, a cycle may start by supplying air and then supply cleaning fluid. This may allow for the supplied air to clean way or dislodge any debris on a device before applying a cleaning fluid. The cleaning fluid and air supplied to an external input port 512, 612 may travel through a fluid distribution network 800 to the plurality of cleaning cavity ports 514, 614 of a cleaning cavity 505 of fixture 700. The pressure with which the cleaning fluid is introduced into the external input ports 512, 612 may result in greater (or lesser) pressure at the cleaning cavity ports 614, which may assist in cleaning the device. Any debris as well as the cleaning fluid and air may pass through a gap created by the inner cleaning cavity walls 520 and 620 that allow for the debris, cleaning fluid, and or air to pass while keeping the device in place. This gap may be referred to as an output opening. The debris, cleaning fluid, and or air may pass to the outer cleaning cavity 535 and then may exit the fixture 700 via an output port 516.

In an example embodiment utilizing the fixture 700, one or more cycles may supply air and/or cleaning fluid. In some embodiments, the entire cleaning process may be referred to as a “cycle” comprising a plurality of other cycles (e.g., sub-cycles) according to the various steps described herein. For example, a cleaning process may include one or more iterations of supplying fluid and/or air to the fixture for a predetermined time (e.g., 20 seconds). In some embodiments, the cleaning process may include two or more iterations of supplying fluid and/or air to the fixture for a predetermined time (e.g., 20 seconds each) with a gap therebetween (e.g., a 3 second delay). In some embodiments, the cleaning process may include alternating between fluid and air supply between adjacent cycles (e.g., 20 seconds of fluid supply, 3 seconds of delay, and 20 seconds of air supply). Various non-limiting examples are described herein. In some embodiments, parameters such as fluid type (e.g., air and ethyl or only air), duration of input, sequencing of inputs (e.g., alternating fluids and/or alternating input locations, such as inputs to top fixture body then inputs to bottom fixture body sequentially), and the like may be controlled by the system.

In an example embodiment, multiple cycles may be performed to clean a device or device case, and multiple cycles may collectively be referred to as a cleaning program. A cycle may include the providing of a cleaning fluid or air as well as a period of time to wait after ceasing to provide the cleaning fluid or air. An example cleaning program includes eight cycles. A first cycle may include providing air for 20 seconds via external input port 512A and then ceasing to supply air and waiting for 3 seconds. A second cycle may include providing fluid for 20 seconds via external input port 512A and then ceasing to supply fluid and waiting for 3 seconds. A third cycle may include providing air for 20 seconds via external input port 512B and then ceasing to supply air and waiting for 3 seconds. A fourth cycle may include providing fluid for 20 seconds via external input port 512B and then ceasing to supply fluid and waiting for 3 seconds. A fifth cycle may include providing air for 20 seconds via external input port 612A and then ceasing to supply air and waiting for 3 seconds. A sixth cycle may include providing fluid for 20 seconds via external input port 612A and then ceasing to supply fluid and waiting for 3 seconds. A seventh cycle may include providing air for 20 seconds via external input port 612B and then ceasing to supply air and waiting for 3 seconds. An eighth cycle may include providing fluid for 20 seconds via external input port 612B and then ceasing to supply fluid and waiting for 3 seconds.

During some or all cycles a vacuum may be applied to first fixture body output port 516 to vacuum out air and fluid provided during a cycle as well as remove any debris from the device and/or device case. The period of time of a cycle that is waited after ceasing to supply air or a cleaning fluid may provide time for the air or cleaning fluid to exit the fixture 700. Alternatively, or additionally, a vacuum may not be applied for all or a portion of a cycle, such as to allow a device and/or device case to be immersed in a cleaning fluid. While each of the cycles may have a duty cycle for providing either air or fluid for a first period of time (e.g., 20 seconds) before ceasing supplying air or fluid for a second period of time (e.g., 3 seconds). Each of the cycles may have the same duty cycle (i.e., an amount or percentage of time for which air or a cleaning fluid is supplied compared to an overall cycle time) or each cycle may vary a duty cycle. Additionally, each cycle may be repeated.

In various embodiments, by providing air and/or a cleaning fluid for one or more cycles, a device and/or a device case may be cleaned, including not only removing debris but also removing 99.99% of any viruses that may be on a device and/or device case.

FIG. 14 illustrates an example testing process 1400 in accordance with some embodiments of the present disclosure.

At operation 1402, a loading tray 114 is indexed to a testing station 160. In various embodiments, an index table may index or move a loading tray with one or more devices and/or device cases into position at a testing station 160. The indexing of the loading tray 114 to a testing station 160 may place the device and/or device cases in place for one or more tests to be conducted by the testing station 160. Alternatively, or additionally, moving the device and/or device cases may be moved to a testing station 160 by an index table 110, one or more conveyors, a robotic arm 116, or the like.

At operation 1404, sensor(s) are aligned. The test station 160 may include one or more sensors that may be used to conduct one or more tests (e.g., microphones, speakers, or the like). The sensors may need to be aligned into a position with regard to one or more of the devices, which may mean moving one or more sensors or reorientating a sensor. In various embodiments, a sensor may need to be attached to a device, such as with one or more electrical connectors, such as in embodiments where a test may be to transfer data to a device or communicate with a device via a wired connection. The alignment may be performed by one or more portions of a testing station 160, by a robotic arm 116, by a user, or any combination of these. In some embodiments, the sensor(s) may be pre-aligned and need not be realigned for every test. For example, the sensor(s) may be oriented towards a location where the trays index to at the testing station, such that each subsequent device or set of devices is rotated into alignment with the sensor(s) without moving the sensor(s) or with minimal movement of the sensor(s).

In various embodiments with devices of earbuds, a sensor may be microphone, and a testing station 160 may align a microphone in relation to an earbud. When not testing, the microphone may be moved out of a way of an index that to allow for the movement of a loading tray. Once a device is in a test ready state, the testing station 160 may position the microphone at a specified distance and orientation in relation to the earbud. If more than one model of earbud is being tested, the microphone(s) may need to be aligned differently for each model of earbud. Once the testing station 160 sensors are aligned, a test sequence involving the sensors may be performed.

In various embodiments, the sensors may be fixed in place and the device(s) may be placed without requiring movement or alignment of sensors. For example, a sensor array of a microphone array including a plurality of microphones may be in a fixed position such that an earbud or pair of earbuds may be placed into position for testing. In various embodiments this may include a portion of the sensor array associated with or directed to receive an audio sound from a first earbud (e.g., a left earbud) and a portion of the sensor array associated with or directed to receive an audio sound from a second earbud (e.g., a right earbud). As the alignment may of the sensor array may be fixed, the placement of the earbuds includes aligning the earbuds with the sensor array. In some embodiments with fixed sensors, the sensors may not need to be aligned and operation 1404 may be omitted.

At operation 1406, a test sequence is performed. The testing station 160 may perform one or more tests, which may each be a test sequence of may collectively be a test sequence. In various embodiments, more than one device and/or device case may be loaded to a testing station 160 at a time and a test sequence may test each of the devices and/or device cases in turn or may test them in parallel.

In various embodiments with a device of an earbud, a test may include having the earbud play a sound, which may be a specific note, a specific series of notes, and/or a song. The earbud may have previously been paired with the cleaning and testing station 100 including the testing station 160. Alternatively, or additionally, the testing station 160 may be electrically connected or coupled to the device. The testing station 160, having been paired, connected, or coupled to the device may transmit one or more test signals to the device. The test signals may include the note(s) or song(s) to be played.

A test for a device may be determined based on the device identifier. The testing station 160 may receive the device identifier or device information associated with the device identifier (e.g., model, etc.) that may allow the testing station 160 to determine one or more tests to perform. In various embodiments of earbuds, the test may be to test a volume across a frequency sweep across a range of frequencies the earbud model is associated with, which may be stored in memory of the testing station 160 or a computing device 180. After determining the one or more tests to be performed, the testing station may perform the tests.

At operation 1408, a successful test is confirmed. The testing station 160, having performed the testing, may receive and record testing information generated by one or more sensors of the testing station 160. The testing information may be compared to predetermined thresholds and/or predetermined ranges associated with the test. The tests, predetermined thresholds, and predetermined ranges may be distinct from other of the same based on the make and the model of the device being tested.

In various embodiments with devices of earbuds, a test may include an earbud generating one or more audio signals that are detected by one or more microphones. The testing station 160 may generate one or more audio signals for the earbud to emit or play. The audio signal may be transmitted from the testing station 160 to the earbud, such as via a wireless connection (e.g., Bluetooth®, etc.). The audio signals may be for a range of frequencies. The test may be associated with both a range of frequencies for which to generate audio signals as well as a volume at which to generate the audio signals. One or more microphones at a known distance away may be expected to receive a predetermined volume over the frequency range. The microphones may receive the emitted audio signal, generate one or more electrical signals in response, and record the electrical signals as test data in a test data object, which may be stored by the testing station 160. The test data may be compared to a volume threshold, which may determine if the earbud is generating the expected audio signals. If the audio signals are as expected, a first grade may be determined. If the audio signal are as expected for some of the test but not others, a second grade may be determined. If not audio signals meet the expected threshold, then yet another grade may be determined. Various embodiments may assign a grade for each test, which may be used to generate an cumulative testing grade. Alternative, a test grade may only be determined for the device without grading each test.

At operation 1410, sensor(s) are moved. At the completion of testing, one or more sensors may be moved away from the device and/or device cases being tested. As noted above, in some embodiments, the sensors may not move at all, such that this step may be omitted in some circumstances. The sensors may be moved to allow for the loading tray 114, device(s), and/or device case(s) to be moved. This may include movement out of the testing station 114. In various embodiments where a device and/or device case was electrically connected or coupled to a testing station 160, the electric connection and/or electric coupling may be undone or remove.

At operation 1412, it is determined if testing was passed. After a grade has been determined for a device, the testing station 116 may determine if the device passed testing. In various embodiments a grade may indicate pass or fail. Alternatively, a grade may be one of a plurality of grade associated with passing testing or a plurality of grades associated with failing testing. Such plurality of grades may be determined based on the performance of a device and/or device case one or more tests.

At operation 1414, if testing was not passed, the device(s) and/or device case(s) are rejected. A rejected device and/or device case may be moved to a reject station 145.

At operation 1416, if testing was passed, the loading tray is indexed. In various embodiments, passing testing may result in the device and/or device case to be moved to an unloading station 170. This may include moving the loading tray 114 from a testing station 160 to by indexing an index table 110, using a robotic arm 116, and/or a user moving the loading tray, device, and/or device case.

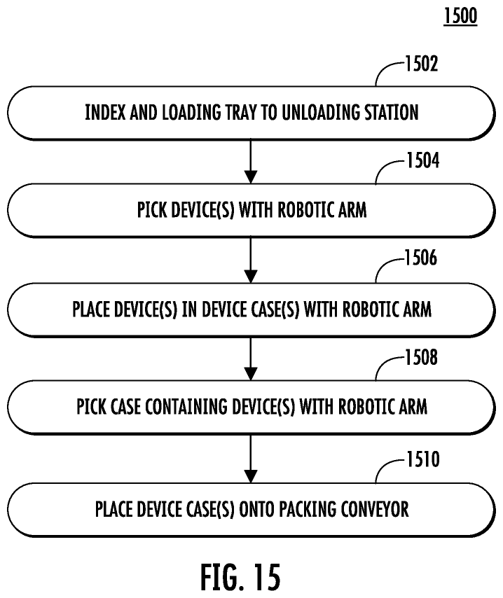

FIG. 15 illustrates an example unloading process 1500 in accordance with some embodiments of the present disclosure.

At operation 1502, a loading tray 114 is indexed to an unloading station 170. The indexing or moving of the loading tray 114 may move a device and/or device case to an unloading station 170 where the device and/or device case may be unloaded from a loading tray 114. The device and/or device case may be placed onto an unloading station 170, which may direct the device and/or device case to one or more areas of the unloading station.

At operation 1504, device(s) are picked with the robotic arm 116. The device may be unloaded with a robotic arm 116, which may pick up or more the device. In some embodiments, multiple robotic arms may be used. In some embodiments, any other apparatus capable of manipulating the various device(s) and/or case(s) may be used.

At operation 1506, device(s) are placed in their device case(s) with robotic arm 116. A robotic arm may open a device case, place the device into the device case, and close the device case.

At operation 1508, device case(s) are picked with the robotic arm 116. The robotic arm may pick put the device case and move it to one or more areas of the unloading station 170. The robotic arm 116 may place the device case into one or more packaging units, which may package the device and/or device case.

At operation 1510, the device case(s) are placed onto a packing conveyor 172. The device cases, which include the device and may be in a packaging unit, may be placed onto a packing conveyor 172. The packing conveyor 172 may convey or move the device case to one or more areas of the unloading station 170 to allow for the device, device case, and/or packaging unit to be packaged. This may include packaging for shipment. In some embodiments, any other device handling equipment may be used.

In various embodiments, the cleaning and testing system 100 may self-diagnose errors and/or track maintenance, such as with the different stations or at the computing device 180.

Each station may include one or more sensors associated with performing an operation described herein. For example, a fluid reservoir in a fluid supply 430 of the cleaning station 150 include one or more sensors to detect a fluid level of a fluid reservoir, fluid flow rate of a fluid in the cleaning station, vacuum level, etc. On a determination that a sensor is not reporting a value in a predetermined operating range, one or more portions of the cleaning and testing station 100 may be operated, cease an operation, or shut off. As another example, an air container of an air supply 440 of a cleaning station may include one or more pressure sensors to sense a pressure of an air container, which may be used to determine when a compressor needs to be run to pressurize the air container if the pressure falls below a predetermined threshold. Additionally, a blow off valve may be operated if the pressure is determined to be above a high threshold.

Additionally, the completion of one or more of the operations described herein may be tracked and evaluated to determine if there is an error. In one embodiment, a cleaning station may be leaking ethyl alcohol from a fluid reservoir. The leak may result in an operation of introducing a cleaning fluid into a fixture failing to be completed. It may be determined that this operation has not been completed, such as with one or more sensors. For example, a flow sensor associated with one or more external input ports of a fixture may indicate no fluid has been introduced. In another example, a flow sensor associated with the output of a fixture may indicate that no fluid is flowing. In yet another example, a computing device 180 may determine that an electrical signal is not reaching a pump, which may be due to a breaker, fuse, or protection relay tripping, and of which might be sensed via one or more relays or due to the shutdown of a pump. In yet another example, the audio test station 160 may indicate that an audio signal is being received by a first microphone but not received by a second microphone, which may indicate a failure of the second microphone.

If an error is determined or detected, an alert may be generated. In various embodiments, each of the stations of the cleaning and testing system 100 may include one or more warning devices, which may include a light, a display, a breaker, or the like that may operate in the event of an alert associated with an error. For example, a light may illuminate in addition to a station ceasing one or more operations. Additionally, or alternatively, the computing device may display one or more alerts and or errors. A user may then be alerted to the error and take actions to address the error.

### Example Computing Device

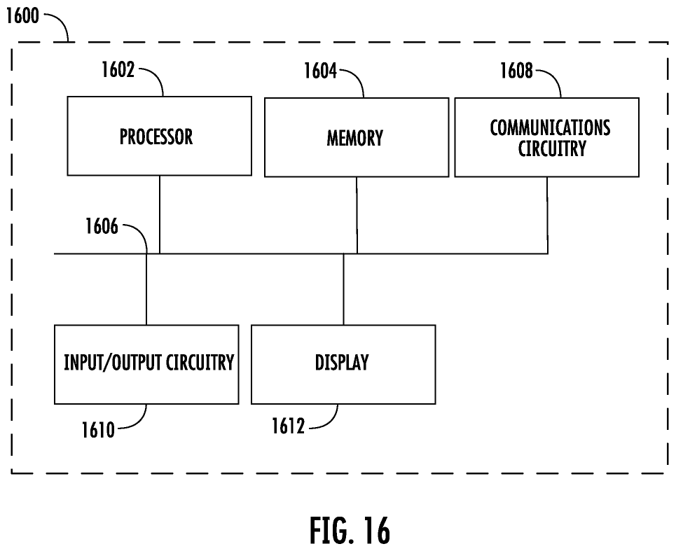

FIG. 16 illustrates an example computing device 1500 in accordance with some embodiments of the present disclosure. Computing device 180 may include one or more components illustrated in the example computing device 1500 of FIG. 16. It is appreciated that various embodiments may also omit one or more components illustrated in FIG. 16. A computing device 180 may include one or more processor 1602, memory 1604, communication circuitry 1608, input/output circuitry 1610, and display 1612, which may be connected via bus 1606.

Computing device 180 may include and be controlled by the processor 1602. Processor 1602 may be embodied in a number of different ways and may, for example, include one or more processing devices configured to perform independently. Additionally, or alternatively, the processor 1602 may include one or more processors configured in tandem via a bus to enable independent execution of instructions, operations, pipelining, and/or multithreading. The use of the term “processor” may be understood to include a single core processor, a multi-core processor, multiple processors internal to the apparatus, and/or remote or “cloud” processors. Further, processor 1602 may be embodied as one or more other processing devices or circuitry. The term circuitry may refer to an entirely hardware embodiment or a combination of hardware and computer program products. Thus, the processor 1602 may be embodied as integrated circuits, application specific integrated circuits (ASICs), field programmable gate arrays (FPGAs), programmable logic arrays (PLAs), hardware accelerators, other circuitry, and/or the like.

Processor 1602, which may be configured to execute instructions for conducting one or more operations, and these instructions may be stored in the memory 1604 or otherwise accessible to the processor. Alternatively, or additionally, the processor 1602 may be configured to execute hard-coded functionality. As such, whether configured by hardware, computer program products, software methods, or by a combination thereof, the processor 1602 may represent an entity (e.g., physically embodied in circuitry) capable of performing operations according to an embodiment of the present disclosure. Alternatively, as another example, when the processor 1602 is embodied as an executor of software instructions, the instructions may specifically configure the processor to perform algorithms and/or operations described herein when the instructions are executed.

Computing device 180 may include memory 1604, which may be non-transitory and may include, for example, one or more volatile and/or non-volatile memories. In other words, for example, the memory 1604 may be an electronic storage device (e.g., a computer readable storage medium). The memory 1604 may be configured to store information, data, content, applications, instructions, or the like, for enabling the cleaning and testing system 100 or a station of a cleaning and testing system 100, including a processor 1602, to carry out various operations in accordance with example embodiments of the present disclosure. In this regard, the memory 1604 may be preconfigured to include computer-coded instructions (e.g., computer program code), and/or dynamically be configured to store such computer-coded instructions for execution by the processor 1602.

Computing device 180 may include a communications circuitry 1608 that may enable the transmission of signals and/or data to other devices (such as, but not limited to, one or more stations or computing device in a station). The communications circuitry 1608 may be any means such as a device or circuitry embodied in either hardware or a combination of hardware and software that is configured to receive and/or transmit a signal and/or data from/to a network and/or any other device, circuitry, or module in communication with the computing device 180 of the cleaning and testing system 100. In this regard, the communications circuitry 1608 may include, for example, a network interface for enabling communications with a wired or wireless communication network. In various embodiments, the communications circuitry may include one or more transmitters to transmit a 4-20 mA signal, a digital signal, a wireless signal, etc. For example, the communications circuitry 1608 may include one or more circuitries, network interface cards, antennae, buses, switches, routers, modems, and supporting hardware and/or software, or any other device suitable for enabling communications via a network. Additionally, or alternatively, the communication interface may include the circuitry for interacting with the antenna(s) to cause transmission of signals via the antenna(s) or to handle receipt of signals received via the antenna(s).

Computing device 180 may include input/output circuitry 1610 that may, in turn, be in communication with the processor 1602 to receive an indication of an input from a user and to provide output to a user. The input/output circuitry 1610 may comprise an interface, a mobile application, a kiosk, or the like. In some embodiments, the input/output circuitry 1610 may also include a keyboard, a mouse, a joystick, a touch screen, touch areas, soft keys, a microphone, a speaker, or other input/output mechanisms. The processor 1602 and/or input/output circuitry 1610 may be configured to control one or more functions of one or more user interface elements through computer program instructions (e.g., software and/or firmware) stored on a memory accessible to the processor (e.g., memory 1604).

Computing device 180 may include a display 1612 that may be in communication with the processor 1602 to display user interfaces (such as, but not limited to, control, progress, and/or results of one or more operations described herein). In various examples of the present disclosure, the display 1612 may include a liquid crystal display (LCD), a light-emitting diode (LED) display, a plasma (PDP) display, a quantum dot (QLED) display, and/or the like.

In various embodiments, the cleaning and testing system 100 may utilize machine learning and/or neural networks, such as with the optical reader of the scanning station 140 for image recognition and processing. The machine learning and/or neural networks may be executed by the computing device 180 or by one of the stations.

Various types of machine learning may be used, including but not limited to supervised learning, unsupervised learning, and reinforcement learning. Such machine learning models may be subjected to and/or implement unsupervised training, supervised training, semi-supervised training, reinforcement learning, deep learning, and/or the like in order to analyze user device data and in turn, determine and utilize relationships for such user device data for fault detection, device analysis, and network analysis according to the various embodiments herein. During training of such a machine learning model (e.g., artificial/convolutional neural network or the like), the model may be iteratively supplied with a plurality of user device data such that the model may be configured to, over time, determine patterns amongst the plurality of parameters, values, and data points defined by the data. Said differently, the machine learning model may be configured to determine a correlation or pattern associated with the values and parameters within the user device data so as to determine a more accurate prediction of health and/or fault information.

In various embodiments, a machine learning model may be trained to address an entire image captured with an optical reader 142. Alternatively, instead of addressing an entire image at once, a machine learning model may be trained to address one or more portions of a captured image, such as the portion of the image associated with a device and/or device case, which may be addressed collectively, separately, or in conjunction. In various embodiments, what the machine learning model addresses may be based on the training data sets that may be used to train the machine learning model. Moreover, a model, including machine learning models, may be created and/or trained to identify and classify relevant portions of images, which may define inflection points and groupings of portions of images around which data is consistent and generally representative and indicative of, for example, damage or wear (e.g., within a predetermined confidence level).

In various embodiments, a machine learning training data set may include historical images, such as those previously captured with an optical reader 142. The historical images may also be from a different cleaning and testing system 100 or a set of predefined images. The machine learning model may include reinforcement learning, and it may continue to learn as additional images or data is generated and/or provided, including images, test results, and/or grades associated with the images.

In some embodiments, a machine learning model may be generated and run in association with a manufacturer, a make, and/or a model of a device and/or device case. In such embodiments, the machine learning model may exclude data related to other devices and/or device cases, including excluding different makes and/or models. Additionally, or alternatively, in some embodiments, a machine learning model may be generated and run in associated with a grading of a device, such that the machine learning model may recognize a device and/or device case (e.g., a serial number), generate a determination of any damage or wear, assign a grade to an image captured with an optical reader 142, and/or perform other operations described herein.

### Non-Limiting Example Embodiments of the Disclosure

Having described various aspects of the innovations, it will be appreciated that various embodiments are described herein. The subject matter described herein includes, without limitation, the following specific embodiments. These embodiments are merely examples and should not be construed as limiting the scope of the disclosure. It will be appreciated that the embodiments in some aspects are freely combinable. In other aspects of the present disclosure, each embodiment is independent from other embodiments described.

Embodiment 1: A fixture configured for cleaning a device, the fixture comprising:
at least one fixture body configured to define an inner cleaning cavity bounded by at least one inner cleaning cavity wall;
wherein the at least one inner cleaning cavity wall defines a plurality of cleaning cavity ports;
wherein the at least one inner cleaning cavity wall defines an output opening; and
wherein the inner cleaning cavity is configured to receive a cleaning fluid via the plurality of cleaning cavity ports and permit the cleaning fluid to exit the inner cleaning cavity via the output opening.

Embodiment 2: The fixture according to Embodiment 1, wherein the at least one fixture body comprises a first fixture body and a second fixture body configured to engage each other;
wherein the at least one inner cleaning cavity wall comprises a first inner cleaning cavity wall defined by the first fixture body and a second inner cleaning cavity wall defined by the second fixture body; and
wherein the inner cleaning cavity comprises a first portion and a second portion, wherein the first inner cleaning cavity wall at least partly bounds the first portion of the inner cleaning cavity and the second inner cleaning cavity wall at least partly bounds the second portion of the inner cleaning cavity.

Embodiment 3: The fixture according to Embodiment 2, wherein the plurality of cleaning cavity ports comprises a first plurality of cleaning cavity ports defined by the first inner cleaning cavity wall of the first fixture body, and a second plurality of cleaning cavity ports defined by the second inner cleaning cavity wall of the second body.

Embodiment 4: The fixture according to Embodiment 3, wherein each of the first plurality of cleaning cavity ports is fluidically connected to an exterior of the fixture via at least one first external input port, and each of the second plurality of cleaning cavity ports is fluidically connected to an exterior of the fixture via at least one second external input port.

Embodiment 5: The fixture according to Embodiment 4, wherein the first fixture body comprises a first fluid distribution network configured to fluidically connect each of the first plurality of cleaning cavity ports with the at least one first external input port, wherein the first fluid distribution network comprises a first input chamber connected to the at least one first external input port and a plurality of first distribution channels, and wherein the plurality of first distribution channels are connected to the first plurality of cleaning cavity ports; and

wherein the second fixture body comprises a second fluid distribution network configured to fluidically connect each of the second plurality of cleaning cavity ports with the at least one second external input port, wherein the second fluid distribution network comprises a second input chamber connected to the at least one second external input port and a plurality of second distribution channels, and wherein the plurality of second distribution channels are connected to the second plurality of cleaning cavity ports.

Embodiment 6: The fixture according to any one of Embodiments 2-5, wherein the output opening of the inner cleaning cavity is defined between the first fixture body and the second figure body in an instance in which the first fixture body and the second fixture body are engaged with each other.

Embodiment 7: The fixture according to any one of Embodiments 2-6, wherein the output opening and each of the plurality of cleaning cavity ports are configured to be smaller than the device to prevent the device from leaving the inner cleaning cavity in the instance in which the first fixture body and the second fixture body are engaged with each other.

Embodiment 8: The fixture according to any one of Embodiments 1-7, wherein the output opening is defined circumferentially around the inner cleaning cavity.

Embodiment 9: The fixture according to any one of Embodiments 1-8, further comprising an output port configured to fluidically couple the output opening to an exterior of the fixture.

Embodiment 10: The fixture according to any one of Embodiments 2-9, wherein the first inner cleaning cavity wall defines a first shape that is configured to match a first half of the device with a clearance between the device and the first inner cleaning cavity wall, and wherein the second inner cleaning cavity wall defines a second shape that is configured to match a second half of the device with a clearance between the device and the second inner cleaning cavity wall.

Embodiment 11: The fixture according to any one of Embodiments 2-10, wherein the first fixture body further comprises one or more first guides and the second fixture body comprises one or more second guides, and wherein the first guides comprise complementary shapes to the associated second guides to laterally align the first fixture body with the second fixture body.

Embodiment 12: The fixture according to any one of Embodiments 2-11, wherein the first fixture body defines a first external wall;
wherein the second fixture body defines a second external wall; and
wherein the first fixture body and second fixture body are configured to be mated to form an airtight seal between the first external wall and second external wall.

Embodiment 13: The fixture according to any one of Embodiments 1-12, wherein the at least one fixture body is further configured to define an outer cavity separate from the inner cleaning cavity and fluidically coupled to the inner cleaning cavity via the output opening.

Embodiment 14: The fixture according to Embodiment 13, wherein the outer cavity is fluidically connected to an exterior of the fixture via an output port.

Embodiment 15: The fixture according to any one of Embodiments 13-14, wherein the outer cavity is configured to surround the inner cleaning cavity along at least one plane.

Embodiment 16: The fixture according to any one of Embodiments 13-15, wherein the at least one fixture body comprises a first fixture body and a second fixture body configured to engage each other at the at least one plane, and wherein the output opening is defined between the first fixture body and the second fixture body.

Embodiment 17: A method for cleaning a device using a fixture, the fixture comprising at least one fixture body configured to define an inner cleaning cavity bounded by at least one inner cleaning cavity wall, wherein the at least one inner cleaning cavity wall defines a plurality of cleaning cavity ports, wherein the at least one inner cleaning cavity wall defines an output opening, wherein the inner cleaning cavity is configured to receive a cleaning fluid via the plurality of cleaning cavity ports and permit the cleaning fluid to exit the inner cleaning cavity via the output opening, the method comprising:
placing the device into the inner cleaning cavity of the fixture; and
cleaning the device by:
   introducing the cleaning fluid into the inner cleaning cavity via each of the plurality of cleaning cavity ports; and
   allowing the cleaning fluid to exit the inner cleaning cavity via the output opening.

Embodiment 18: The method for cleaning a device using a fixture according to Embodiment 17, further comprising:
introducing air into the fixture via each of the plurality of cleaning cavity ports; and
allowing the air to leave the inner cleaning cavity via the output opening.

Embodiment 19: The method for cleaning a device using a fixture according to Embodiment 18, wherein allowing the air to leave the inner cleaning cavity via the output opening comprises:

extracting, via a vacuum generated by a pump, air from the inner cleaning cavity.

Embodiment 20: The method for cleaning a device using a fixture according to any one of Embodiments 17-19, wherein the cleaning fluid includes ethyl alcohol.

Embodiment 21: The method for cleaning a device using a fixture according to any one of Embodiments 17-20, wherein introducing the cleaning fluid is for a first period of time, and wherein allowing the cleaning fluid to exit the inner cleaning cavity occurs after an end of the first period of time.

Embodiment 22: The method for cleaning a device using a fixture according to any one of Embodiments 17-21, wherein the at least one fixture body includes a first fixture body and a second fixture body.

Embodiment 23: The method for cleaning a device using a fixture according to Embodiment 22, wherein the first fixture body comprises one or more guides, and wherein the second fixture body comprises one or more guide cavities, wherein each of the one or more guide cavities is configured to mate to one of the guides of the first fixture body.

Embodiment 24: The method for cleaning a device using a fixture according to Embodiment 23 further comprising:

mating, prior to the cleaning, the first fixture body to the second fixture body utilizing the one or more guides and one or more guide cavities.

Embodiment 25: The method for cleaning a device using a fixture according to any one of Embodiments 22-24,
wherein the at least one inner cleaning cavity wall includes a first inner cleaning cavity wall and a second inner cleaning cavity wall;
wherein the plurality of cleaning cavity ports includes a first plurality of cleaning cavity ports defined by the first inner cleaning cavity wall and a second plurality of cleaning cavity ports defined by the second inner cleaning cavity wall;
wherein the first fixture body comprises the first inner cleaning cavity wall and at least one external input port of the first fixture body fluidically connected to the first plurality of cleaning cavity ports;
wherein the second fixture body comprises the second inner cleaning cavity wall and at least one external input port of the second fixture body fluidically connected to the second plurality of cleaning cavity ports.

Embodiment 26: The method for cleaning a device using a fixture according to Embodiment 25, wherein introducing the cleaning fluid into the inner cleaning cavity via each of the plurality of cleaning cavity ports comprises introducing cleaning fluid via the at least one external input port of the first fixture body and via the at least one external input port of the second fixture body.

Embodiment 27: A system for cleaning devices, the system comprising:
a fixture comprising:
   at least one fixture body configured to define an inner cleaning cavity bounded by at least one inner cleaning cavity wall;
   at least one external input port;
   wherein the at least one inner cleaning cavity wall defines a plurality of cleaning cavity ports;
   wherein the at least one inner cleaning cavity wall defines an output opening;
   wherein the inner cleaning cavity is configured to receive a cleaning fluid via the plurality of cleaning cavity ports and permit the cleaning fluid to exit the inner cleaning cavity via the output opening;
a fluid supply connected to the at least one external input port; and
an air supply connected to the at least one external input port.

Embodiment 28: The system for cleaning devices according to Embodiment 27 further comprising:

a cleaning station configured to connect the air supply and the fluid supply to the fixture.

Embodiment 29: The system for cleaning devices according to any one of Embodiments 27-28, wherein the fixture comprises at least one fixture body including a first fixture body and a second fixture body, wherein the first fixture body and the second fixture body are configured to engage each other;
wherein the cleaning station is further configured to:
   engage the first fixture body with the second fixture body; and
   connect the air supply and the fluid supply with the fixture only after engagement of the first fixture body with the second fixture body.

Embodiment 30: The system for cleaning devices according to any one of Embodiments 27-29 further comprising:

a pairing station configured to pair to at least one device.

Embodiment 31: The system for cleaning devices according to Embodiment 30, wherein the pairing station comprises at least one actuator to actuate one or more buttons associated with a device to pair the device to the system.

Embodiment 32: The system for cleaning devices according to any one of Embodiments 27-31 further comprising:

a scanning station configured to scan a device identifier of a device.

Embodiment 33: The system for cleaning devices according to Embodiment 30 further comprising:

at least one robotic arm configured to position a device identifier in view of an optical reader.

Embodiment 34: The system for cleaning devices according to any one of Embodiments 27-33 further comprising:

a testing station configured to test a device.

Embodiment 35: The system for cleaning devices according to any one of Embodiments 27-34, wherein the device comprises an audio device;
wherein the testing station comprises one or more microphones;
wherein the testing station is configured to:
   generate at least one audio signal to be emitted by the audio device;
   transmit the at least one audio signal to the audio device;
   generate, by the one or more microphones and in response to the at least one audio signal being emitted by the audio device, at least one electrical signal associated with the audio device; and
   generate a grading of the audio device based on the at least one electrical signal.

Embodiment 36: The system for cleaning devices according to any one of Embodiments 27-35 further comprising:

one or more rotary index tables configured to rotate a device to one or more stations.

Embodiment 37: The system for cleaning devices according to Embodiment 36, wherein the one or more stations include one or more of a pairing station, a scanning station, and a testing station.

Embodiment 38: A method for cleaning and testing a device, the method comprising:
cleaning the device using a fixture, the fixture comprising at least one fixture body configured to define an inner cleaning cavity bounded by at least one inner cleaning cavity wall, wherein the at least one inner cleaning cavity wall defines a plurality of cleaning cavity ports, wherein the at least one inner cleaning cavity wall defines an output opening, wherein the inner cleaning cavity is configured to receive a cleaning fluid via the plurality of cleaning cavity ports and permit the cleaning fluid to exit the inner cleaning cavity via the output opening, wherein cleaning the device using the fixture comprises:
   placing the device into the inner cleaning cavity of the fixture;
   introducing the cleaning fluid into the inner cleaning cavity via each of the plurality of cleaning cavity ports; and
   allowing the cleaning fluid to exit the inner cleaning cavity via the output opening;
testing the device, after cleaning the device and via at least one testing station, to generate a grading of the performance of the device.

Embodiment 39: The method for cleaning and testing a device according to Embodiment 38, wherein allowing the cleaning fluid to exit the inner cleaning cavity via the output opening comprises introducing air from an air supply into the inner cleaning cavity via each of the plurality of cleaning cavity ports.

Embodiment 40: The method for cleaning and testing a device according to any one of Embodiments 38-39, wherein the at least one fixture body includes a first fixture body and second fixture body, wherein the first fixture body comprises one or more guides, wherein the second fixture body comprises one or more guide cavities, wherein each of the one or more guide cavities is configured to mate to one of the guides of the first fixture body;
wherein cleaning the device using the fixture further comprises:
   mating, after the placing the device into the inner cleaning cavity of the fixture and before connecting a fluid supply to the fixture, the first fixture body to the second fixture body utilizing the one or more guides and one or more guide cavities.

Embodiment 41: The method for cleaning and testing a device according to any one of Embodiments 38-40, wherein the device is an audio device, wherein testing station comprises one or more microphones, and wherein testing the device further comprises:
generating at least one audio signal to be emitted by the audio device;
transmitting the at least one audio signal to the audio device;
generating, by the one or more microphones and in response to the at least one audio signal being emitted by the audio device, at least one electrical signal associated with the audio device; and
generating the grading of the audio device based on the at least one electrical signal.

Embodiment 42: The method for cleaning and testing a device according to any one of Embodiments 38-41 further comprising:

pairing, before cleaning the device, the device with a pairing station.

Embodiment 43: The method for cleaning and testing a device according to any one of Embodiments 38-42 further comprising:

scanning a device identifier of the device with a scanning station.

Embodiment 44: The method for cleaning and testing a device according to Embodiment 43 wherein scanning a device identifier comprises:
positioning the device identifier of the device in a view of an optical reader with at least one robotic arm; and
scanning, with the optical reader, the device identifier.

Embodiment 45: The method for cleaning and testing a device according to any one of Embodiments 38-44 further comprising:
generating an acceptance indication based on the grading; and
sorting the device based on the acceptance indication.

Embodiment 46: The method for cleaning and testing a device according to Embodiment 45, wherein the sorting the device utilizes at least one robotic arm to sort the device to one of a plurality of locations.

### CONCLUSION

Although exemplary embodiments have been described above, implementations or embodiments of the subject matter and the operations described herein can be implemented in other types of digital electronic circuitry, computer software or program, firmware, or hardware, including the structures disclosed in this specification and their structural equivalents, or in combinations of one or more of them.

Embodiments of the subject matter described herein may be implemented as one or more computer programs, i.e., one or more modules of computer program instructions, encoded on computer storage medium for execution by, or to control the operation of, information/data processing apparatus. Alternatively, or in addition, the program instructions can be encoded on an artificially-generated propagated signal, e.g., a machine-generated electrical, optical, or electromagnetic signal, which is generated to encode information/data for transmission to suitable receiver apparatus for execution by an information/data processing apparatus. A computer storage medium can be, or be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially-generated propagated signal. The computer storage medium can also be, or be included in, one or more separate physical components or media (e.g., multiple CDs, disks, or other storage devices).

The operations described herein can be implemented as operations performed by an information/data processing apparatus on information/data stored on one or more computer-readable storage devices or received from other sources.

The processes described herein can be performed by one or more programmable processors executing one or more computer programs to perform actions by operating on input information/data and generating output. Processors suitable for the execution of a computer program include, by way of example, both general and special purpose microprocessors, and any one or more processors of any kind of digital computer. Generally, a processor will receive instructions and information/data from a read-only memory or a random access memory or both. The essential elements of a computer are a processor for performing actions in accordance with instructions and one or more memory devices for storing instructions and data. Generally, a computer will also include, or be operatively coupled to receive information/data from or transfer information/data to, or both, one or more mass storage devices for storing data, e.g., magnetic, magneto-optical disks, or optical disks. However, a computer need not have such devices. Devices suitable for storing computer program instructions and information/data include all forms of non-volatile memory, media, and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

The term “data processing apparatus” as used above encompasses all kinds of apparatus, devices, and machines for processing data, including by way of example a programmable processor, a computer, a system on a chip, or multiple ones, or combinations, of the foregoing. The apparatus may include special purpose logic circuitry, e.g., an FPGA (field programmable gate array) or an ASIC (application-specific integrated circuit). The apparatus may also include, in addition to hardware, code that creates an execution environment for the computer program in question, e.g., code that constitutes processor firmware, a protocol stack, a repository management system, an operating system, a cross-platform runtime environment, a virtual machine, or a combination of one or more of them. The apparatus and execution environment may realize various different computing model infrastructures, such as web services, distributed computing, and grid computing infrastructures.

Computer software or computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A computer program can be stored in a portion of a file that holds other programs or information/data (e.g., one or more scripts stored in a markup language document), in a single file dedicated to the program in question, or in multiple coordinated files (e.g., files that store one or more modules, sub-programs, or portions of code). A computer program can be deployed to be executed on one computer or on multiple computers that are located at one site or distributed across multiple sites and interconnected by a communication network.

While this specification contains many specific implementation details, these should not be construed as limitations on the scope of any disclosures or of what may be claimed, but rather as descriptions of features specific to particular embodiments of particular disclosures. Certain features that are described herein in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

Thus, particular embodiments of the subject matter have been described. Other embodiments are within the scope of the following claims. In some cases, the actions recited in the claims can be performed in a different order and still achieve desirable results. In addition, the processes depicted in the accompanying figures do not necessarily require the particular order shown, or sequential order, to achieve desirable results. In certain implementations, multitasking and parallel processing may be advantageous.

1. A method for cleaning an earbud, the method comprising:
placing an earbud into a cleaning cavity of a fixture; and
supplying a cleaning fluid comprising alcohol to the cleaning cavity to clean the earbud.
